(19) United States
(12) Patent Application Publication
(10) Pub. No.: US 2012/0009261 A1
(43) Pub. Date: Jan. 12, 2012

(54) NOVEL GASTRO-RETENTIVE DOSAGE FORMS

(75) Inventor: RAMESH SESHA, Windsor, NJ (US)

(73) Assignee: Grünenthal GmbH, Aachen (DE)

(21) Appl. No.: 13/176,798

(22) Filed: Jul. 6, 2011

Related U.S. Application Data
(60) Provisional application No. 61/399,045, filed on Jul. 6, 2010.

(51) Int. Cl. A61K 9/00; A61K 31/485; A61P 25/04; A61K 31/197

(52) U.S. Cl. 424/484; 424/400; 514/561; 514/282

(57) ABSTRACT

A gastro-retentive pharmaceutical dosage form of a therapeutically effective amount of at least one GABA Analog, at least one opioid, and at least one pharmaceutically acceptable excipient wherein the said dosage form is retained in the stomach for at least four hours and is suitable for formulating for once daily or twice daily administration. Further provided is a method of treating a disorder by administering to a patient in need thereof, a gastro-retentive pharmaceutical dosage form of a therapeutically effective amount of at least one GABA Analog, at least one opioid, and at least one pharmaceutically acceptable excipient wherein the said dosage form is retained in the stomach for at least four hours and is suitable for once daily or twice daily administration. The opioid is either in slow release form or in immediate release form.

### PRIORITY CLAIM

This application claims priority of U.S. Provisional Patent Application No. 61/399,045 filed on Jul. 6, 2010, the contents of which are incorporated herein by reference.

### FIELD OF THE INVENTION

The present invention is related to gastro-retentive pharmaceutical dosage forms comprising a GABA Analog, an opioid and the method of using such dosage forms that are retained in stomach for at least four hours and are suitable for twice daily or once daily administration to treat a disorder in mammal.

### BACKGROUND OF THE INVENTION

Gamma (γ)-Aminobutyric acid (GABA) is a neurotransmitter in the mammalian central nervous system and is implicated in number of disease pathways. Pregabalin, a gamma-aminobutyric acid (GABA) analogue, is an anticonvulsant drug which is used as an adjunct therapy for partial seizures, for neuropathic pain, and in generalized anxiety disorder. Pregabalin was designed as a more potent successor to gabapentin and it is marketed by Pfizer under the trade name Lyrica®. Recent studies have shown that pregabalin is effective at treating chronic pain in disorders such as fibromyalgia and spinal cord injury. Gabapentin, is another GABA analogue similar to Pregabalin and was initially synthesized to mimic the chemical structure of the neurotransmitter gamma-aminobutyric acid (GABA), but is not believed to act on the same brain receptors. Its exact mechanism of action is unknown, but its therapeutic action on neuropathic pain is thought to involve voltage-gated N-type calcium ion channels.

GABA analogs such as pregabalin and gabapentin have been approved as immediate release dosage forms that require frequent administration to treat patients. However, this need for frequent dosing can frequently lead to errors in administration and inability to maintain desirable concentration in the plasma which in turn are detrimental to patient compliance and the therapeutic objectives, particularly if the condition is chronic pain or pain related condition. Hence there is an unmet medical need to have a gastro-retentive pharmaceutical dosage form for the administration of GABA Analogs, such as pregabalin and gabapentin, in combination with opioids such as morphine, oxycodone, etc.

However, designing once daily dosage forms of pregabalin or gabapentin, even as single drug presents several challenges; For example, pregabalin is not absorbed uniformly in the gastrointestinal (GI) tract. Studies have shown pregabalin and gabapentin are absorbed in the small intestine and the ascending colon and pregabalin has a very narrow absorption window of around six hours. Similarly, the absorption of immediate release gabapentin occurs relatively slowly with the peak plasma concentration occurring approximately 2 to 3 hours after dosing. The oral bioavailability of gabapentin is dose-dependent, with approximately 60% bioavailability for a dose in the range of 300-400 mg, but with only 35% bioavailability for a dose of 1600 mg. Thus there is unmet need for long acting dosage forms of GABA Analogs such as pregabalin and gabapentin etc.

GABA Analog drugs such as Pregabalin and Gabapentin require a gastro-retentive delivery system to optimize the therapeutic benefits. Drug absorption from gastrointestinal tract is a complex procedure and is subject to many variables. It has been reported that the extent of gastrointestinal tract drug absorption is related to contact time with the small intestinal mucosa. Gastro-retentive system can remain in the gastric region for several hours and therefore significantly prolong the gastric residence time of drugs. Many approaches have been reported in the literature for the formulation of gastro-retentive system viz. mucoadhesion, swelling, floatation, sedimentation, expansion and modified shape systems.

Slow release pharmaceutical dosage forms, including gastro-retentive delivery systems, are well known and provide distinct advantages for delivery of drugs which act optimally at certain levels of plasma concentration over extended periods of time. Slow release systems may also avoid the presence of ineffective or toxic levels of drugs which result from periodic administration of immediate release dosage forms which provide high initial levels of drug but may leave only ineffectively small amounts of drugs in the plasma near the end of the administration periods (i.e. cycles) prior to subsequent administration of drug. These are particularly suited for chronic conditions such as pain and pain related conditions by providing drugs in a sustained released manner that only requires administration either once or twice daily instead of every four to eighteen hours as may be indicated for a particular drug.

In U.S. Pat. Nos. 4,571,333 and 4,803,079, Hsias and Kent disclose the use of controlled release naproxen formulations and disclose the use of controlled release naproxen sodium formulations. Therapeutic blood peak levels of naproxen are not achieved promptly by these formulations and take greater than 6 hours to be achieved, as indicated by the maximum concentrations (Cₘₐₓ) disclosed therein. Similarly, the U.S. Pat. No. 5,508,042 provide Controlled release oxycodone formulations for the treatment of pain. U.S. Pat. No. 5,614,218 provides An oral controlled release pharmaceutical preparation in the form of a tablet, capsule or sachet containing a plurality of coated particles comprising a therapeutically effective amount of a salt of morphine coated with a barrier membrane providing a controlled, preferably pH-independent, release of morphine. U.S. Pat. No. 6,285,887 disclose the controlled release oral formulations containing tramadol. U.S. Pat. No. 7,410,965 provides a delayed release pharmaceutical formulation containing 1-dimethylamino-3-(3-methoxyphenyl)-2-methylpentan-3-ol or a pharmaceutically acceptable salt thereof in a matrix. U.S. Pat. No. 5,601,842 disclose a tablet containing tramadol and a matrixing agent with a viscosity between 3,000 and 150,000 mPa in a 2% aqueous solution at 20° C.

Further U.S. Pat. No. 5,811,126 discloses a controlled release pharmaceutical composition containing tramadol and comprising sodium alginate, C₂ to C₅₀ edible hydrocarbon derivative with melting point range from 25° C. to 90° C. and divalent salt to cross link the alginate. In vivo performance from these formulations is not available. U.S. Pat. Nos. 5,639,476 and 5,580,578 disclose controlled release dosage forms containing a substrate containing tramadol, said substrate being coated with a plasticized aqueous dispersion of ammonio-methacrylate copolymer having low content of quaternary ammonium groups and a permeability enhancing pore former, said coating being cured for about 24 to about 60 hours to stabilize said formulation. U.S. Pat. No. 5,955,104 discloses a delayed release tramadol formulation consisting of pellets in a water soluble capsule or in a tablet compressed from said pellets, each pellet having (a) a substantially inert core; (b) an active ingredient layer over the inert core and containing (i) tramadol particles, (ii) with a binder for adhering said tramadol particles over said inert core, and optionally (iii) a pharmaceutically acceptable, inner adjuvant; and (c) a delay coating for retarding the release of tramadol consisting principally of mixtures of ethyl cellulose and shellac.

Novel slow release formulations have also been developed using high amylose starch, and, in particular, recent advances have been made using cross-linked high amylose starch. For example, U.S. Pat. No. 5,456,921, U.S. Pat. No. 5,616,343, U.S. Pat. No. 6,284,273, U.S. Pat. No. 6,419,957 and U.S. Pat. No. 6,607,748, describe solid controlled release oral pharmaceutical dosage units in the form of tablets comprising a dry powder of a pharmaceutical product and a dry powder of cross-linked high amylose starch in which the cross-linked high amylose starch includes a mixture of about 10-60% by weight of amylopectin and about 40-90% amylose.

U.S. Pat. No. 3,490,742 disclose a binder-disintegrant comprising non-granular amylose. This material is prepared either by fractionating starch or by dissolving granular high amylose starch in water at an elevated temperature. No controlled release properties are disclosed. U.S. Pat. No. 5,108,758 disclose an oral delayed release composition comprising an active compound and glassy amylose. European patent application No. EP-A-499,648 discloses a tablet excipient. More particularly, they disclose a starch binder and/or filler useful in manufacturing tablets, pellets, capsules or granules. U.S. Pat. No. 7,410,965 and US. Pat. Application No. 20050136110 provides a delayed release pharmaceutical composition of 1-dimethyl-lamino-3(3-methoxyl-phenyl)-2-methyl-pentan-3-ol. There have been suggestions of extended and controlled release formulations of tramadol HCl U.S. Pat App. No. 2003/0143270, U.S. Pat. No. 6,254,887, US. Pat. App. No. 2001/0036477 (Miller et al.) U.S. Pat. No. 6,326,027, U.S. Pat. No. 5,591,452 and European Patent 1 190 712. Though there is one drug purported to be a twice-a day in clinical trials, there is not a single slow release tapentadol formulations on the market, let alone drugs that are once-a-day formulations nor are there any formulations that lower the side effects. US. Pat. App. 20070269511 claims a solid pharmaceutical composition containing pregabalin is described. The composition includes a matrix forming agent and a swelling agent and is suitable for once daily oral administration and it includes a matrix forming agents include mixtures of polyvinyl acetate and polyvinylpyrrolidone. US Pat App. No 20070269511 claims gastro-retentive once daily solid pharmaceutical compositions that comprise pregabalin. Similarly US. Pat. App. No. 20060159743 claims a gastric retentive dosage form of gabapentin that is capable of administration in once-daily or twice daily dosing regimens. WO2009067703 discloses a pharmaceutical dosage form comprising a slow release tapentadol and a second active agent such as tapentadol to treat pain and pain related disorder. WO2010025931 claims combination comprising tapentadol hydrochloride and an anti-epileptic and method of treating pain using such combination. However, there is no prior art of a gastro-retentive pharmaceutical dosage form comprising a GABA Analog, and an opioid, that is suitable for once daily and twice daily administration. Similarly there are no reports of gastro-retentive pharmaceutical dosage form comprising a slow release GABA analog and an opioid wherein the opioid is either in slow release form or in immediate release form.

Accordingly, the instant invention discloses a pharmaceutical dosage form comprising a therapeutically effective amount of at least one GABA Analog, at least one opioid, and at least one pharmaceutically acceptable excipient wherein the said dosage form is retained in the stomach for at least four hours and is suitable twice or once daily administration. Further, the present inventor while working on the analgesic compositions have surprisingly found gastro-retentive dosage forms and methods of treating moderate to severe painful conditions associated with diabetic neuropathy, rheumatoid arthritis, osteoarthritis and the like, by administering to a subject in need thereof, a pharmaceutical dosage form comprising of 5-2000 mg of a therapeutically effective amount of at least one GABA Analog and 1-1000 mg of an opioid wherein the said dosage form is retained in the stomach for at least four hours and is suitable once daily or twice daily administration so as to provide better pain management.

This invention is advantageous as there will be a decreased dosing of the active ingredient, with substantial patient compliance and sustained period of pain relief.

### BRIEF DESCRIPTION OF THE INVENTION

The present invention provides a pharmaceutical dosage form comprising a therapeutically effective amount of at least one GABA Analog, at least one opioid, and at least one pharmaceutically acceptable excipient wherein the said dosage form is retained in the stomach for at least four hours and is suitable for once daily or twice daily administration.

The present invention further provides a method of treating a disorder by administering a pharmaceutical a dosage form comprising a therapeutically effective amount of at least one GABA Analog, at least one opioid, and at least one pharmaceutically acceptable excipient wherein the said dosage form is retained in the stomach for at least four hours and is suitable for once daily or twice daily administration.

The invention further provides a pharmaceutical dosage form comprising a therapeutically effective amount of at least one GABA Analog, at least one opioid, and at least one pharmaceutically acceptable excipient, wherein the said opioid is in immediate release form and the dosage form is retained in the stomach for at least four hours and is suitable for once daily or twice daily administration.

The invention further provides a pharmaceutical dosage form comprising a therapeutically effective amount of at least one GABA Analog, at least one opioid, and at least one pharmaceutically acceptable excipient, wherein the said GABA Analog and the said opioid are in slow release forms and the dosage form is retained in the stomach for at least four hours and is suitable for once daily or twice daily administration.

The present invention further provides a method of treating a disorder by administering a pharmaceutical a dosage form comprising a therapeutically effective amount of at least one GABA Analog, at least one opioid, and at least one pharmaceutically acceptable excipient, wherein the said opioid is in immediate release form and the dosage form is retained in the stomach for at least four hours and is suitable for once daily or twice daily administration.

The present invention further provides a method of treating a disorder by administering a pharmaceutical a dosage form comprising a therapeutically effective amount of at least one GABA Analog, at least one opioid, and at least one pharmaceutically acceptable excipient, wherein the said GABA Analog and the said opioid are in slow release forms and the dosage form is retained in the stomach for at least four hours and is suitable for once daily or twice daily administration.

The present invention further provides a pharmaceutical dosage form comprising: a) a core comprising at least two release layers wherein a first release layer comprising at least one GABA Analog, and a second release layer comprises at least one opioid and at least one pharmaceutically acceptable excipient, b) a coat comprising the said core, wherein the dosage form is retained in the stomach for at least four hours and is suitable for once daily or twice daily administration.

The present invention further provides a method of treating a disorder by administering a pharmaceutical a dosage form comprising: a) a core comprising at least two release layers wherein a first release layer comprising at least one GABA Analog, and a second release layer comprises at least one opioid and at least one pharmaceutically acceptable excipient; and b) a coat comprising the said core, wherein the dosage form is retained in the stomach for at least four hours and is suitable for once daily or twice daily administration.

The present invention further provides a pharmaceutical a dosage form comprising a) a compressed core comprising at least two release layers wherein a first release layer comprising at least one GABA Analog dispersed in a slow-release matrix; and a second release layer comprising an opioid dispersed in a second release matrix; b) at least one permeable membrane covering comprising the said core; c) an encapsulating coat and the said dosage form is suitable for once daily or twice daily administration.

The present invention further provides a method of treating a disorder by administering a pharmaceutical a dosage form comprising a) a compressed core comprising at least two release layers wherein a first release layer comprising at least one GABA Analog dispersed in a slow-release matrix; and a second release layer comprising an opioid dispersed in a second release matrix; b) at least one permeable membrane covering comprising the said core; c) an encapsulating coat and the said dosage form is suitable for once daily or twice daily administration.

The present invention further provides a pharmaceutical dosage form comprising a therapeutically effective amount of at least one GABA Analog and at least one opioid and at least one pharmaceutically acceptable excipient wherein the dosage form exhibits an in-vitro dissolution profile wherein

;between 10% and 40% of GABA Analog released between 0 and about 2 hours of measurement,

;between about 30% and 60% of GABA Analog is released between 2 and about 7 hours of the measurement,

;between about 50% and 80% of GABA Analog is released between 7 and about 12 hours of measurement, and

;between about 80% and 100% of GABA Analog is released after about 20 hours of measurement

The present invention further provides a pharmaceutical dosage form comprising a therapeutically effective amount of at least one GABA Analog and at least one opioid and at least one pharmaceutically acceptable excipient, having a dissolution rate in vitro when measured with USP apparatus Type 1 at 100 rpm in 50 mM sodium phosphate buffer at pH 6.8, wherein

;from about 5% to about 30% of GABA Analog released after 1 hour;

;from about 15% to about 40% of GABA Analog released after 2 hours;

;from about 20% to about 50% of GABA Analog released after 4 hours,

;from about 30% to about 70% of GABA Analog released after 8 hours;

;from about 40% to about 90% of GABA Analog released after 12 hours;

;from about 50% to about 100% of GABA Analog released after 16 hours;

;from 60% to about 100% of GABA Analog released after 24 hours.

The present invention further provides a pharmaceutical dosage form comprising a therapeutically effective amount of at least one GABA Analog and at least one opioid and at least one pharmaceutically acceptable excipient, having a dissolution rate in vitro when measured with USP apparatus Type 1 at 100 rpm in 50 mM sodium phosphate buffer at pH 6.8, wherein

;from about 10% to about 25% of GABA Analog released after 1 hour;

;from about 15% to about 30% of GABA Analog released after 2 hours;

;from about 25% to about 40% of GABA Analog released after 4 hours,

;from about 40% to about 55% of GABA Analog released after 8 hours;

;from about 60% to about 75% of GABA Analog released after 12 hours;

;from about 70% to about 90% of GABA Analog released after 16 hours;

;from about 90% to about 100% of GABA Analog released after 24 hours.

The present invention further provides a pharmaceutical dosage form comprising a therapeutically effective amount of at least one GABA Analog, at least one opioid, and at least one pharmaceutically acceptable excipient, wherein the said dosage form is retained in the stomach for at least four hours and the said opioid is selected from a group consisting of alfentanil, Axomadol, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, faxeladol, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tapentadol, and, tramadol, and the said dosage form is suitable for once daily or twice daily administration.

The present invention further provides a method of treating pain by administering a pharmaceutical dosage form comprising a therapeutically effective amount of at least one GABA Analog, at least one opioid, and at least one pharmaceutically acceptable excipient, wherein the said dosage form is retained in the stomach for at least four hours and the said opioid is selected from a group consisting of alfentanil, Axomadol, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, faxeladol, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tapentadol, and, tramadol, and the said dosage form is suitable for once daily administration and the said dosage form is suitable for once daily or twice daily administration.

### BRIEF DESCRIPTION OF THE DRAWINGS

FIG. 1 is the depiction of the dissolution profile of pregabalin in a gastro-retentive fixed combination of pregabalin and tapentadol (Example 1 and 2).

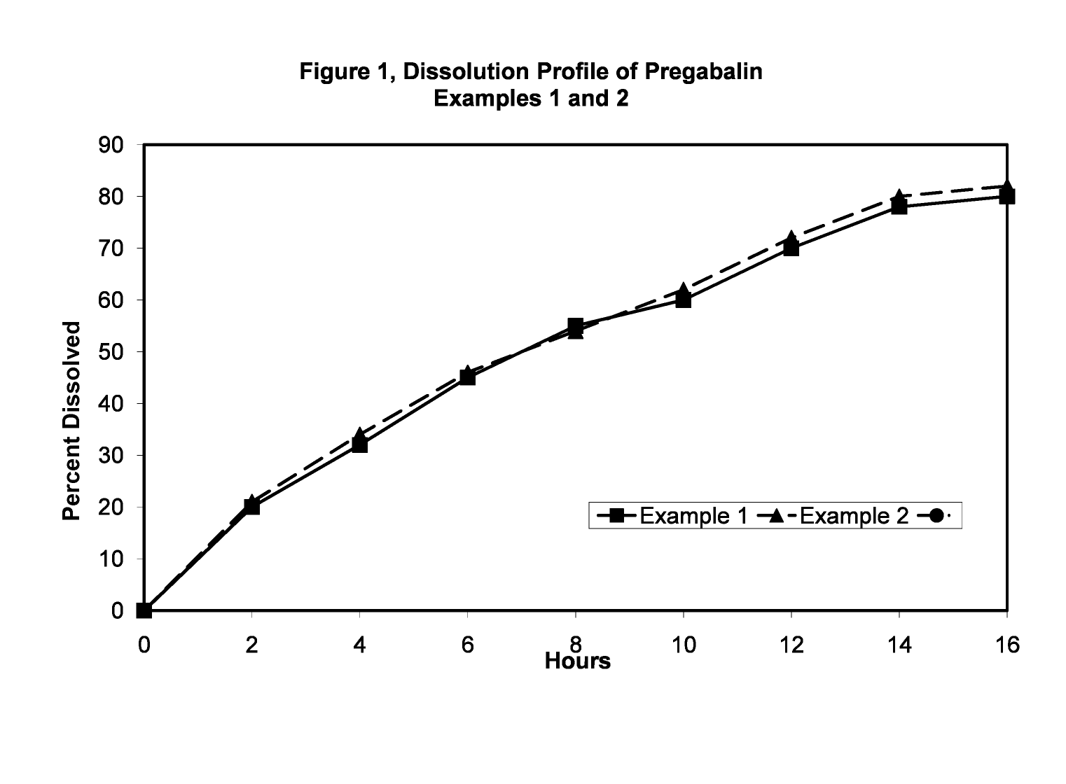

FIG. 2 is the depiction of the dissolution profile of pregabalin in a gastro-retentive fixed combination of pregabalin and tapentadol (Example 8, 9 and 10)

FIG. 3 is the depiction of the mean plasma concentration of pregabalin in a gastro-retentive fixed combination of pregabalin and tapentadol (Example 1 and Reference Example 1)

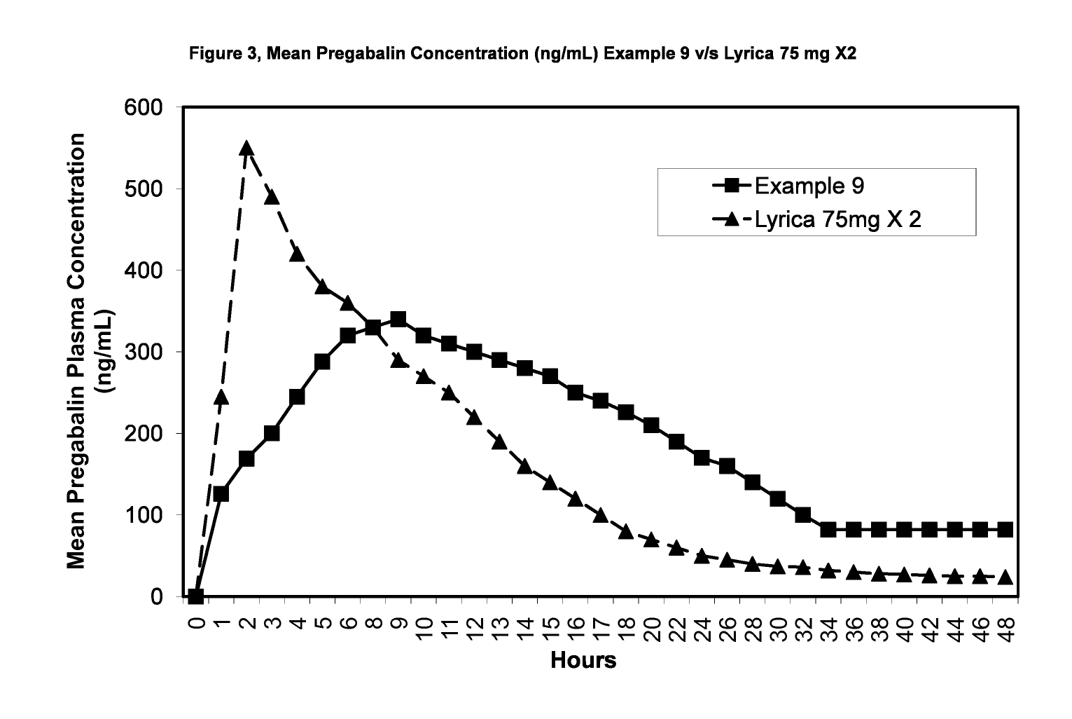

FIG. 4 is the depiction of the dose proportionality of pregabalin in a gastro-retentive fixed combination of pregabalin and tapentadol (Example 8, 9 and 10)

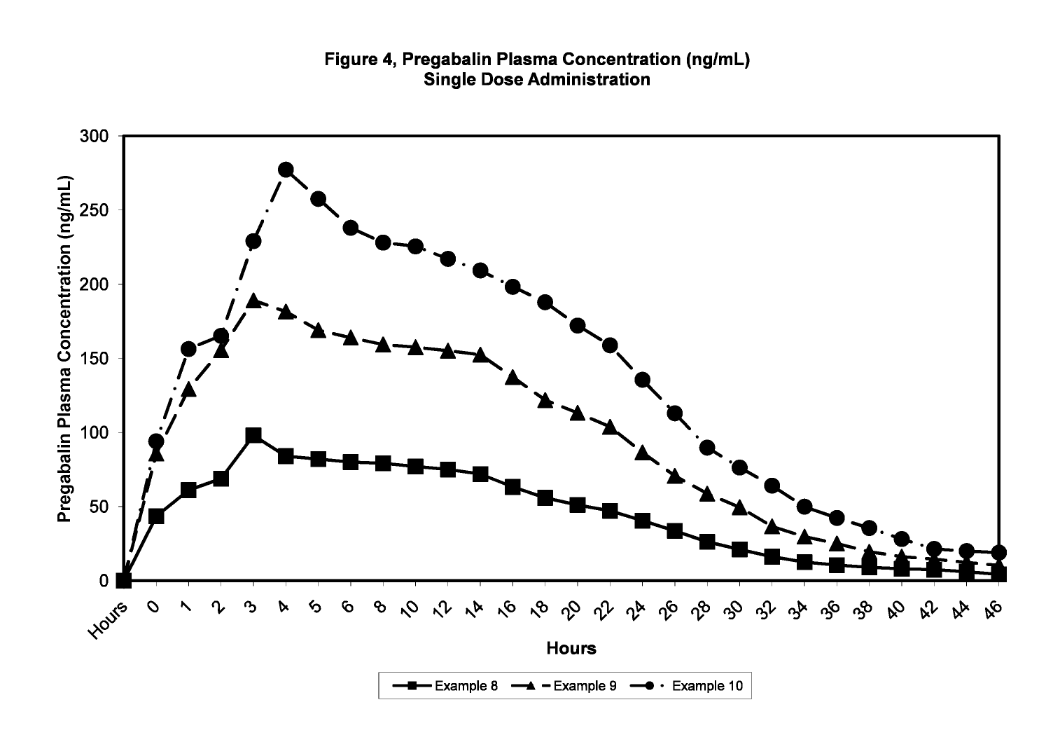

FIG. 5 is the depiction of the Steady State Tapentadol Concentration (ng/mL) in a gastro-retentive fixed combination of pregabalin and tapentadol (Example and Reference Example)

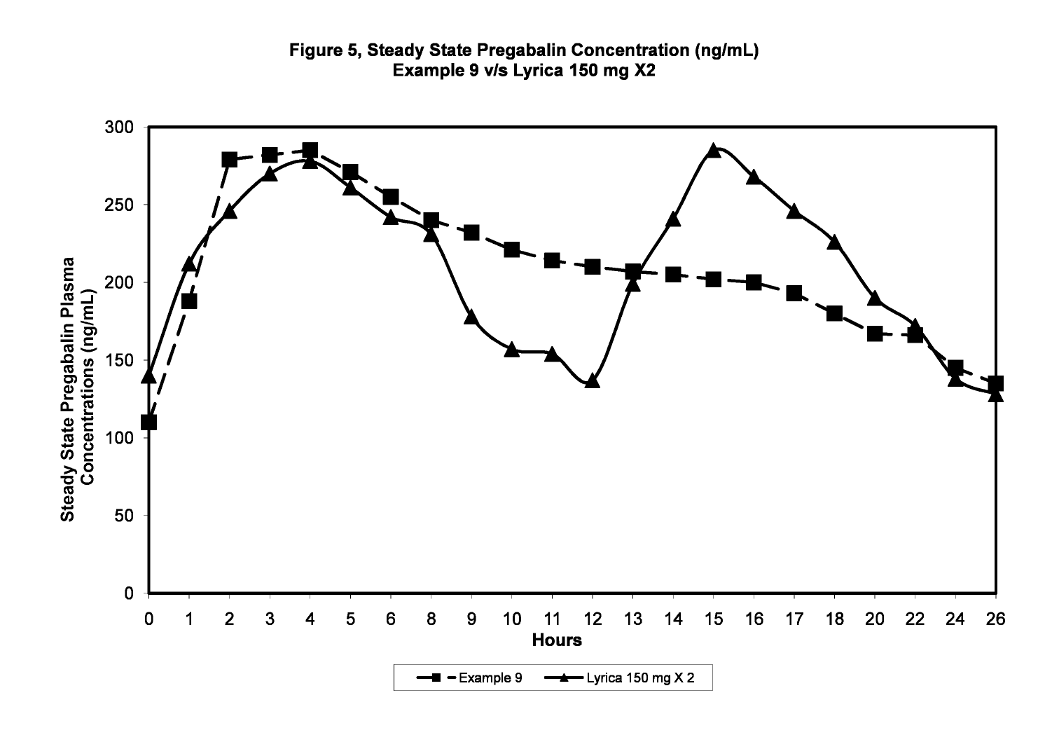

FIG. 6 is the depiction of the mean pain scores of a gastro-retentive fixed combination of pregabalin and tapentadol (Example 8) in comparison with tapentadol and pregabalin alone.

### DETAILED DESCRIPTION OF THE INVENTION

The term “administration or ingestion” used herein means administration of dose of a formulation containing an active ingredient administered to a patient or subject.

The term “amylose” as used herein means a linear polymer of glucose and made of several thousand glucose units.

The term “binding agent” as used in this specification, refers to any conventionally known pharmaceutically acceptable binder such as polyvinyl pyrrolidone, hydroxypropyl cellulose, hydroxyethyl cellulose, hydroxypropyl methylcellulose, ethyl cellulose, polymethacrylate, polyvinyl alcohol, waxes and the like. Mixtures of the aforementioned binding agents may also be used. The preferred binding agents are water soluble materials such as polyvinyl pyrrolidone having a weight average molecular weight of 25,000 to 3,000,000. The binding agent may comprise approximately about 0 to about 40% of the total weight of the core and preferably about 3% to about 15% of the total weight of the core. In one embodiment, the use of a binding agent in the core is optional.

The term “bioequivalence or bioequivalent” is defined as the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. There being about a 90% or greater probability that the bioavailability (AUC) of a GABA Analog as determined by standard methods is about 80 to about 125% of the second orally administrable dosage form comprising the same dose of a GABA Analog and that there is a about 90% or greater probability that the maximum blood plasma concentration (Cₘₐₓ) of a GABA Analog as measured by standard methods is about 80 to about 125% of the second orally administrable dosage form.

The term “clinical effect” as used herein as clinical efficacy with respect to pain experienced by study subjects measured using a suitable scale, for example; WOMAC global score, Likert-scale, or VAS score.

The term “controlled-release” as used herein is defined to mean a substantially gradual rate of release of the drug in the first once daily controlled-release dosage form or the at least one means for controllably releasing the in a substantially controlled manner per unit time in-vivo. The rate of release of the drug is controlled by features of the dosage form and/or in combination with physiologic or environmental conditions rather than by physiologic or environmental conditions alone.

The term “controlled-release dosage forms” or dosage forms which exhibit a “controlled-release” of a GABA Analog or an Opioid as used herein is defined to mean dosage forms administered once daily that release drug at a relatively constant rate and provide plasma concentrations of the active drug that remain substantially invariant with time within the therapeutic range of the active drug over about a 24-hour period.

The term “cross linked amylose” as used herein means amylase units linked with one another.

The term “Covering” as herein means a pharmaceutically acceptable retarding covering such as a film or a coating.

The term “candidate for sustained release” encompasses all the characteristics of a drug which make it a candidate for formulating it into an extended release fashion like a short elimination half life and consequent dosing of more than once a day, a single dose product given in an extended fashion to achieve better clinical results and avoid side effects associated with an immediate release etc.

The term “delayed-release dosage forms” or dosage forms which exhibit a “delayed-release” of the drug as used herein is defined to mean dosage forms administered once daily that do not substantially release drug immediately following administration but at a later time. Delayed-release dosage forms provide a time delay prior to the commencement of drug-absorption. Such dosage forms will desirably be coated with a delayed-release coat.

The term “dosage form” as used herein is defined to mean a solid oral pharmaceutical preparation or system in which doses of medicine or active drug are included. A dosage form will desirably comprise, for example, at least one slow release dosage form including various slow release forms such as, osmosis controlled-release dosage form, erosion controlled-release dosage form, dissolution controlled-release dosage form, diffusion controlled-release dosage form, controlled-release matrix core, controlled-release matrix core coated with at least one release-slowing coat, enteric coated dosage form, one sustained dosage, dosage form surrounded by at least one delayed-release coat, capsules, minitablets, caplets, uncoated micro particles, micro particles coated with release-slowing coat, micro particles coated with delayed-release coat or any combination thereof. Within the context of this application, the dosage forms described herein mean a dosage form as defined above comprising an effective amount of a GABA Analog and an opioid for treating a patient in need thereof.

The term “effective amount” as used herein means a dosage which is sufficient in order for the treatment of the patient to be effective compared with no treatment.

The term “enhanced absorption dosage forms” or dosage forms which exhibit an “enhanced absorption” of the drug as used herein is defined to mean dosage forms that when exposed to like conditions, will show higher release and/or higher absorption of the drug as compared to other dosage forms with the same or higher amount of drug.

The term “extended release material” as present in the inner solid particulate phase and the outer solid continuous phase refers to one or more hydrophilic polymers and/or one or more hydrophobic polymers and/or one or more other type hydrophobic materials, such as, for example, one or more waxes, fatty alcohols and/or fatty acid esters. The “extended release material” present in the inner solid particulate phase may be the same as or different from the “extended release material” present in the outer solid continuous phase.

The term “extended-release dosage forms” or dosage forms which exhibit an “extended release” of drug as used herein is defined to mean dosage forms administered once daily that release drug slowly, so that plasma concentrations of the drug are maintained at a therapeutic level for an extended period of time such that the sustained-release dosage form provides therapeutic benefit over a 24-hour period.

The term “FDA guidelines” refers to the guidance, Guidance for Industry Bioavailability and Bioequivalence Studies approved by the US Food and Drug Administration at the time of filing of this patent application. The Guidance for Industry Bioavailability and Bioequivalence Studies for Orally Administered Drug Products General Considerations, U.S. Department of Health and Human Services, Food and Drug Administration, Center for Drug Evaluation and Research (CDER) along with accompanying label of Sep. 8, 2005 is herewith incorporated in its entirety.

The term “GABA Analog or GABA Analogue” as used herein is defined to mean at least one form of a GABA Analog that bind to calcium channel alpha-2-delta receptor and these include, but not limited to tiagabine, pregabalin, gabapentin etc. They include respective GABA Analog base, the individually optically active enantiomers of a GABA Analog, such as for example, (+) or (−) forms of a GABA Analog, racemic mixtures thereof, active metabolites, pharmaceutically acceptable salts thereof, such as for example, acid addition or base addition salts of a GABA Analog. Acids commonly employed to form acid addition salts are inorganic acids, such as for example, hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutylate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutylate, citrate, lactate, g-hydroxybutylate, glycolate, tartrate, methanesulfonate, propanesulfonate, naphthalene-1-sulfonate, napththalene-2-sulfonate, mandelate and the like. Base addition salts include those derived from inorganic bases, such as for example, ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like.

The term “hydrophilic polymers” as used in this specification include, but are not limited to hydroxypropylmethylcellulose, hydroxypropylcellulose, sodium, carboxymethyl-cellulose, carboxymethylcellulose calcium, ammonium alginate, sodium alginate, potassium alginate, calcium alginate, propylene glycol alginate, alginic acid, polyvinyl alcohol, povidone, carbomer, potassium pectate, potassium pectinate, etc

The term “hydrophobic polymers” as used in this specification include, but are not limited, to ethyl cellulose, hydroxyethylcellulose, ammonio methacrylate copolymer (Eudragit RL™ or Eudragit RS™), methacrylic acid copolymers (Eudragit L™ or Eudragit S™), methacrylic acid-acrylic acid ethyl ester copolymer (Eudragit L 100-5™), methacrylic acid esters neutral copolymer (Eudragit NE 30D™), dimethylaminoethylmethacrylate-methacrylic acid esters copolymer (Eudragit E 100™), vinyl methyl ether/malefic anhydride copolymers, their salts and esters (Gantrez™) etc.

The term “immediate release coat”, as used herein, is defined to mean a coat, which has substantially or appreciably no influence on the rate of release of a GABA Analog or an opioid from the dosage form in-vitro or in-vivo. The excipients comprising the immediate release coat have no substantial slow release, swelling, erosion, dissolution, or erosion and swelling properties, which means that the composition of the coat has no substantial influence on the rate of release of the a GABA Analog or an opioid.

The term “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

The term “medicament” as used herein means a pharmaceutical composition suitable for administration of the pharmaceutically active compound to a patient.

The term “mean maximum plasma concentration” (Cₘₐₓ) as used herein means the arithmetic mean of maximum plasma concentration of a GABA Analog or an opioid.

The term “mean plasma concentration” as used herein means the arithmetic mean blood plasma concentration of a GABA Analog or opioid.

The term “modified-release dosage forms” or dosage forms which exhibit a “modified-release” of the drug as used herein is defined to mean dosage forms whose drug release characteristics of time course and/or location are designed to accomplish therapeutic or convenience objectives not offered by an immediate-release dosage forms. Modified-release dosage forms or dosage forms are typically designed to provide a quick increase in the plasma concentration of the drug which remains substantially constant within the therapeutic range of the drug for at least a 24-hour period. Alternatively, modified-release dosage forms will desirably be designed to provide a quick increase in the plasma concentration of the drug, which although may not remain constant, declines at rate such that the plasma concentration remains within the therapeutic range for at least a 24-hour period.

The term “multiparticulate” or “microparticle” as used herein is defined to mean a plurality of drug-containing units, such as for example microspheres, spherical particles, microcapsules, particles, micro particles, granules, spheroids, beads, pellets, or spherules.

The term “opioids or opiates” as used herein means any entity that brings out biological response by acting on opioid receptors. These include but not limited to opioid agonists useful in the present invention include, but are not limited to, alfentanil, Axomadol, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, faxeladol, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tapentadol, and, tramadol, mixtures or salts of any of the foregoing.

The term “pain and pain related conditions” as used herein is defined as any pain due to a medical conditions including but not limited to neuropathic pain, osteoarthritis, rheumatoid arthritis, fibromyalgia, and back, musculoskeletal pain, Enclosing spondylitis, juvenile rheumatoid arthritis, migraines, dental pain, abdominal pains, ischemic pain, postoperative pain or because of an anesthetic or surgical contrition.

The term “passage” includes an aperture, orifice, bore, hole, weakened area or a credible element such as a gelatin plug that erodes to form an osmotic passage for the release of the drug from the dosage form.

The term “pharmaceutically acceptable derivative” means various pharmaceutical equivalent isomers, enantiomers, salts, hydrates, polymorphs, esters etc of a GABA Analog or an opioid.

The term “prevention of a disease” as used herein is defined as the management and care of an individual at risk of developing the disease prior to the clinical onset of the disease. The purpose of prevention is to combat the development of the disease, condition or disorder, and includes the administration of the active compounds to prevent or delay the onset of the symptoms or complications and to prevent or delay the development of related diseases, conditions or disorders.

The term “swellable polymer,” as used herein, refers to a polymer that will swell in the presence of a fluid. It is understood that a given polymer may or may not swell when present in a defined drug formulation.

The term “slow-release” here applies to any release formulation that is other than an immediate release wherein the release of the active ingredient is slow in nature. This includes various terms used interchangeably in the pharmaceutical context like extended release, delayed release, sustained release, controlled release, timed release, specific release, prolonged release and targeted release etc.

The term “sustained-release dosage forms” or dosage forms which exhibit a “sustained-release” of the drug as used herein is defined to mean dosage forms administered once daily that provide a release of the drug sufficient to provide a therapeutic dose after administration, and then a gradual release over an extended period of time such that the sustained-release dosage form provides therapeutic benefit over a 24-hour period.

The term “treatment of a disease” as used herein means the management and care of a patient having developed the disease, condition or disorder. The purpose of treatment is to combat the disease, condition or disorder. Treatment includes the administration of the active compounds to eliminate or control the disease, condition or disorder as well as to alleviate the symptoms or complications associated with the disease, condition or disorder.

The term “twice daily oral pharmaceutical composition” as used herein is defined as any formulation administered two times a day to a patient in need thereof.

The term “therapeutically effective amount” means an amount that elicits a biological response in a mammal including the suboptimal amount.

Other hydrophobic materials which may be employed in the inner solid particulate phase and/or outer solid continuous phase include, but are not limited, to waxes such as beeswax, carnauba wax, microcrystalline wax, and ozokerite; fatty alcohols such as cetostearyl alcohol, stearyl alcohol; cetyl alcohol myristyl alcohol etc; and fatty acid esters such as glyceryl monostearate, glycerol monooleate, acetylated monoglycerides, tristearin, tripalmitin, cetyl esters wax, glyceryl palmitostearate, glyceryl behenate, hydrogenated castor oil, etc.

Suitable polymers for use in the present dosage forms may be linear, branched, dendrimeric, or star polymers, and include synthetic hydrophilic polymers as well as semi-synthetic and naturally occurring hydrophilic polymers. The polymers may be homopolymers or copolymers, if copolymers, either random copolymers, block copolymers or graft copolymers. Synthetic hydrophilic polymers useful herein include, but are not limited to: polyalkylene oxides, particularly poly(ethylene oxide), polyethylene glycol and poly(ethylene oxide)-poly(propylene oxide) copolymers; cellulosic polymers; acrylic acid and methacrylic acid polymers, copolymers and esters thereof, preferably formed from acrylic acid, methacrylic acid, methyl acrylate, ethyl acrylate, methyl metbacrylate, ethyl methacrylate, and copolymers thereof, with each other or with additional acrylate species such as aminoethyl acrylate; maleic anhydride copolymers; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol), poly(N-vinyl lactams) such as poly(vinyl pyrrolidone), poly(N-vinyl caprolactam), and copolymers thereof polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol and polyoxyethylated glucose; polyoxazolines, including poly(methyloxazoline) and poly(ethyloxazoline); polyvinylamines; polyvinylacetates, including polyvinylacetate per se as well as ethylene-vinyl acetate copolymers, polyvinyl acetate phthalate, and the like, polyimines, such as polyethyleneimine; starch and starch-based polymers; polyurethane hydrogels; chitosan; polysaccharide gums; zein; and shellac, ammoniated shellac, shellac-acetyl alcohol, and shellac n-butyl stearate.

The present invention discloses a pharmaceutical dosage form comprising a therapeutically effective amount of at least one GABA Analog, at least one opioid, and at least one pharmaceutically acceptable excipient wherein the said dosage form is retained in the stomach for at least four hours and is suitable for once daily or twice daily administration. Exemplary GABA Analogs include pregabalin and gabapentin and exemplary opioids include morphine, oxycodone, tramadol, tapentadol, axomadol and faxeladol.

The present invention further provides a pharmaceutical dosage form comprising: a) a core comprising at least two release layers wherein a first release layer comprising at least one GABA Analog dispersed in a slow-release matrix comprising cross-linked high amylose starch; and a second release layer comprising at least one opioid and, dispersed in a second-release matrix; and b) a coat comprising the said core. Exemplary GABA Analogs include pregabalin and gabapentin and exemplary opioids include morphine, oxycodone, tramadol, tapentadol, axomadol and faxeladol.

The core includes at least one matrix and the GABA Analog is slowly released from the matrix. In a specific embodiment, one matrix of the core is a cross-linked high amylose starch prepared according to the standard procedures and described in detail below. The core is formed by mixing the constituents layers and compressing them into a compressed core. The weight of the core could be from about 10% to about 80% of the tablet weight. In the preferred embodiments, the core is from 26% to 33% depending on the amount of a GABA Analog used to make the tablets. The GABA Analog could be from about 10% to about 90% of the total composition. In specific embodiment, GABA Analog amount in a first release layer is on or around 50% of the total GABA Analog present in the tablet and Opioid is on or about 100% of the total opioid present in the dosage form. For example 75 mg in a 150 mg GABA Analog (Pregabalin) dosage or about 50% of a 150 mg GABA Analog (Gabapentin) dosage formulation and 100 mg of tapentadol. In another embodiment of this invention 50 mg in a 100 mg GABA Analog (Gabapentin) dosage or about 50% in a 150 mg GABA Analog (Gabapentin) dosage formulation. GABA Analog is present at levels ranging from about 1 to about 90 wt. % of the total weight of the core, preferably from about 10 to about 70 wt. % of the total composition of the first release layer, more preferably from about 20 to about 60 wt. % of the total composition of the first release layer, and probably most often between about 30 to about 50 wt. % of the total composition of the first release layer. Opioid (Tapentadol) is present from about 10% to about 90% of second release layer.

The core also includes at least one matrix and an opioid is slowly released from the matrix. In a specific embodiment, one matrix of the core is a cross-linked high amylose starch prepared according to the standard procedures and described in detail below. The core is formed by mixing the constituents layers and compressing them into a compressed core. The weight of the core could be from about 10% to about 80% of the tablet weight. In the preferred embodiments, the core is from 26% to 33% depending on the amount of an opioid used to make the tablets. An opioid could be from about 10% to about 90% of the total composition. In specific embodiment, Opioid amount in a first release layer is on or around 50% of the total Opioid present in the tablet. For example 50 mg in a 100 mg opioid (tapentadol) dosage or about 50% of a 150 mg GABA Analog (Gabapentin) dosage formulation and 150 mg of a GABA analog. Opioid is present at levels ranging from about 1 to about 90 wt. % of the total weight of the core, preferably from about 10 to about 70 wt. % of the total composition of the first release layer, more preferably from about 20 to about 60 wt. % of the total composition of the first release layer, and probably most often between about 30 to about 50 wt. % of the total composition of the first release layer.

At least one matrix of the core is cross-linked high amylose starch and it makes up between about 10% and about 90% by weight of the first release layer. In one particular embodiment, the first release layer totals about 140 mg, of which about 75 mg is cross linked amylase and GABA Analog is about 75 mg thus the matrix makes up about 49 weight percent of the first release layer. However the ratio of the matrix of the first release layer to the active ingredient of the first release layer (w/w) is between about 0.1 and about 10, or between about 0.5 and about 5, or between about 1 and about 4, or between about 1 and about 3 and about 1.5 and about 2.5. The first release layer as envisaged in the present invention may optionally include a pharmaceutically acceptable carrier or vehicle flavoring agents; coloring agents; binders; preservatives; lubricants, starch, fillers, glidants, surfactants and the like known to those skilled in the art and are found, for example, in Remington's Pharmaceutical Sciences, 14.sup.th Ed. (1970).

The second release layer of the core includes a physical mixture of polyvinyl acetate and polyvinylpyrrolidone and optionally the active pharmaceutical ingredient(s) of the first release layer. The second layer, prepared by dry compression in a preferred embodiment, can also include a cross-linked high amylose starch. In a particular embodiment described below, polyvinylpyrrolidone making up about 45% by weight of the second layer. The second layer has about 23% of xanthan gum. Opioid is present from about 30% to about 70% by weight of second release layer

The present invention discloses a pharmaceutical composition comprising a GABA Analog, at least one opioid and at least one pharmaceutically acceptable excipient and the dosage form remains in the stomach for at least four hours. According to the invention, the composition preferably contains a therapeutically effective amount of a GABA Analog and therapeutically effective amount of an opioid or a pharmaceutically acceptable salt thereof, wherein the GABA Analog is suitably in the range of from 5 to 800 mg, especially 50, 100, 200, 300, 400 to 1000 mg per dosage unit and Opioid is present from about 1 mg to about 1000 mg the said dosage form is suitable for once daily or twice daily administration. The exact dosage depends on the opioid used in the dosage form. For example; tapentadol could be from about 50 mg to about 150 mg in a dosage while oxycodone could be from about 25 mg to about 100 mg in a dosage form.

The present invention further provides a solid dosage form comprising: a) a core comprising at least two release layers wherein a first release layer comprising at least one GABA Analog dispersed in a slow-release matrix comprising cross-linked high amylose starch; and a second release layer comprising at least one opioid and b) a coat comprising the said core.

The present invention further provides a solid dosage form comprising: a) a core comprising at least two release layers wherein a first release layer comprising at least one GABA Analog dispersed in a slow-release matrix comprising cross-linked high amylose starch; and a second release layer comprising at least one opioid dispersed in a second release matrix, wherein the said second release layer comprises a physical mixture of polyvinyl acetate and polyvinylpyrrolidone, and b) a coat comprising the said core.

The present invention further provides a pharmaceutical dosage form comprising a) a core comprising at least two release layers wherein a first release layer comprising at least one GABA Analog dispersed in a slow-release matrix comprising cross-linked high amylose starch; and a second release layer comprising at least one opioid dispersed in a second release matrix; and b) a coat comprising the said core, wherein between 10% and 30% per hour of GABA Analog initially present at 0 hours, is released between 0 and 2 hours when tested in vitro using a USP Type I apparatus in 50 mM phosphate, pH 6.8, and stirring between 50 and 150 rpm

The present invention further provides a pharmaceutical a dosage form comprising a) a compressed core comprising at least two release layers wherein a first release layer comprising at least one GABA Analog dispersed in a slow-release matrix; and a second release layer comprising an opioid in a second release matrix; b) at least one permeable membrane covering comprising the said core; c) an encapsulating coat comprising the membrane coated core wherein between 10% and 30% per hour of GABA Analog initially present at 0 hours, is released between 0 and 2 hours when tested in vitro using a USP Type I apparatus in 50 mM phosphate, pH 6.8, and stirring between 50 and 150 rpm.

The present invention further provides a solid dosage form comprising: a core comprising a compressed first release layer comprising cross-linked high amylose starch having a GABA Analog, or a salt thereof, embedded therein; and a second release layer comprising at least one opioid in a second release matrix. More preferably, the dosage form comprises from about 5 mg to 500 mg of a GABA Analog dispersed in a first release layer comprising a cross-linked high amylose starch; and a second release layer comprising from about 1 mg to about 500 mg at least one opioid in a second release matrix.

The present invention further provides a solid dosage form comprising: a core comprising a compressed first release layer comprising cross-linked high amylose starch having a GABA Analog, or a salt thereof, embedded therein; and a second release layer comprising at least one opioid in a second immediate release matrix. More preferably, the dosage form comprises from about 5 mg to 500 mg of a GABA Analog dispersed in a first release layer comprising a cross-linked high amylose starch; and a second release layer comprising from about 1 mg to about 500 mg at least one opioid in a second immediate release matrix.

The present invention further provides a slow release tablet comprising: a core comprising a compressed first release layer comprising cross-linked high amylose starch having a GABA Analog, or a salt thereof, embedded therein; and a second release layer comprising a physical mixture of polyvinyl acetate, polyvinylpyrrolidone, a binder, an opioid; and wherein: the ratio of the first release/second release layer (w/w) is between about 0.2 and 0.6.

The present invention further provides a solid dosage form for use for a period of every four hours, or every six hours, every eight hours, every twelve hours, or every eighteen hours, or twenty-four hours, the formulation comprising a compressed core comprising a first release layer comprising a GABA Analog dispersed in a first slow-release matrix comprising cross-linked high amylose starch, and a second release layer comprising at least one opioid in a second slow-release matrix and a coat comprising the said core.

The invention discloses a slow release tablet comprising: a) a core comprising at least two release layers wherein a first release layer comprising at least one GABA Analog dispersed in a slow-release matrix comprising cross-linked high amylose starch; and a second release layer comprising a physical mixture of polyvinyl acetate, polyvinylpyrrolidone, a binder, at least one opioid; and wherein: the ratio of the first release layer/second release layer (w/w) is between about 0.2 and 0.6; the ratio of the a GABA Analog in the first release layer to the opioid in the second release layer is between about 0.7 and about 1, b) a coat comprising said core

The invention discloses a slow release tablet comprising; a) a core comprising at least two release layers wherein a first release layer comprising at least one GABA Analog dispersed in a slow-release matrix comprising cross-linked high amylose starch; and a second release layer comprising a physical mixture of polyvinyl acetate, polyvinylpyrrolidone, a binder, at least one opioid; and wherein: the ratio of the first release layer/second release layer (w/w) is between about 0.2 and 0.6; the ratio of polyvinyl acetate/polyvinylpyrrolidone (w/w) is between about 6:4 and 9:1, b) a coat comprising said core.

In at least one embodiment of the invention, includes a solid dosage formulation comprising a GABA analog and is retained in stomach for at least four hours for releasing GABA analog thereof over an extended period of time, the formulation comprises: a) a core comprising at least two release layers wherein a first release layer comprising at least one GABA Analog dispersed in a slow-release matrix comprising cross-linked high amylose starch; and a second release layer comprising at least one opioid in a second slow release matrix, b) a coat comprising the said core, wherein the said formulation comprises from about 5 to about 800 mg of a GABA Analog and from about 2.5 mg to about 500 mg of opioid.

In at least one embodiment of the invention, includes a solid dosage formulation comprising a GABA analog and is retained in stomach for at least four hours for releasing GABA analog thereof over an extended period of time, the formulation comprises: a) a compressed core comprising at least two release layers wherein a first release layer comprising at least one GABA Analog dispersed in a slow-release matrix comprising cross-linked high amylose starch; and a second release layer optionally comprising GABA Analog in a second slow release matrix b) a coat comprising the said core, wherein the said first layer comprises from about 5 mg to about 800 mg of a GABA analog and second layer comprises from about 2.5 mg to about 500 mg of opioid.

In at least one embodiment of the invention, includes a solid dosage formulation comprising a GABA analog and is retained in stomach for at least four hours for releasing GABA analog thereof over an extended period of time, the formulation comprises: a) a compressed core comprising at least two release layers wherein a first release layer comprising at least one opioid dispersed in a slow-release matrix comprising cross-linked high amylose starch; and a second release layer comprising at least one GABA Analog in a second slow release matrix, b) a coat comprising the said core, wherein the said first layer comprises from about 2.5 mg to about 500 mg of an Opioid and second layer comprises from about 5 mg to about 800 mg of a GABA analog.

The dosage form comprising a therapeutically effective amount of a GABA Analog, at least one opioid and at least one pharmaceutically acceptable excipient may be a bilayer composition that delivers GABA Analog and an opioid over at least twelve hours. The bilayer composition comprises at least one layer that releases an opioid as a rapid-release portion. The pharmaceutical dosage form comprising a therapeutically effective amount of a GABA Analog, an opioid and at least one pharmaceutically acceptable excipient, also comprises a second layer adjacent the first layer defining a sustained release portion that comprises either a GABA alone or a GABA Analog and an opioid, such as tapentadol or oxycodone, and cross-linked high amylose starch as a controlled release excipient. It is understood by a person skilled in art that a bilayer composition of the invention is representative and exemplary. A bilayer dosage form may take a variety of shapes and forms, including tablets, caplets or ovoid, and may be coated or uncoated. The preferred form is a tablet.

In at least one embodiment of the invention, includes a dosage form comprising: a) a core comprising at least two release layers wherein a first release layer comprising at least one GABA Analog dispersed in a slow-release matrix comprising cross-linked high amylose starch; and a second release layer comprising at least one opioid or a GABA analog dispersed in a second release matrix; and a third release layer optionally comprising either a GABA analog or an opioid, and b) a coat comprising the said core.

In at least one embodiment of the invention, includes a dosage form comprising: a) a core comprising at least two release layers wherein a first release layer comprising at least one GABA Analog dispersed in a slow-release matrix comprising cross-linked high amylose starch; and a second release layer comprising at least one opioid or a GABA analog dispersed in a second release matrix; and a third release layer optionally comprising an ANDA antagonist, and b) a coat comprising the said core.

In at least one embodiment of the invention, includes a dosage form comprising: a) a core comprising at least two release layers wherein a first release layer comprising at least one GABA Analog dispersed in a slow-release matrix comprising cross-linked high amylose starch; and a second release layer comprising at least one opioid dispersed in a second release matrix; and a third release layer, wherein one of the layers release layer comprises a physical mixture of polyvinyl acetate, polyvinylpyrrolidone, and b) a coat comprising the said core.

In at least one embodiment of the invention, includes a dosage form comprising: a) a core comprising at least three release layers wherein a GABA analog or an Opioid is dispersed in at least one of the release layers, and b) a coat comprising the said core.

In at least one embodiment of the invention, includes a solid dosage formulation comprising a GABA analog and an opioid for release thereof over an extended period of time, the formulation comprises: a) a compressed core comprising at least two release layers wherein a first release layer comprising at least one GABA Analog dispersed in a slow-release matrix comprising cross-linked high amylose starch; and a second release layer comprising an opioid in a second release matrix; and a third release layer, and b) a coat comprising the said core, wherein the said opioid is selected from a group consisting of alfentanil, Axomadol, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, faxeladol, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tapentadol, and, tramadol,

In at least one embodiment of the invention, includes a solid dosage formulation comprising a GABA analog and an opioid for release thereof over an extended period of time, the formulation comprises: a) a compressed core comprising at least two release layers wherein a first release layer comprising at least one GABA Analog dispersed in a slow-release matrix comprising cross-linked high amylose starch; and a second release layer comprising an opioid in a second release matrix; and a third release layer, and b) a coat comprising the said core, wherein the said opioid is Axomadol,

In at least one embodiment of the invention, includes a solid dosage formulation comprising a GABA analog and an opioid for release thereof over an extended period of time, the formulation comprises: a) a compressed core comprising at least two release layers wherein a first release layer comprising at least one GABA Analog dispersed in a slow-release matrix comprising cross-linked high amylose starch; and a second release layer comprising an opioid in a second release matrix; and a third release layer, and b) a coat comprising the said core, wherein the said opioid is tapentadol.

In at least one embodiment of the invention, includes a solid dosage formulation comprising a GABA analog and an opioid for release thereof over an extended period of time, the formulation comprises: a) a compressed core comprising at least two release layers wherein a first release layer comprising at least one GABA Analog dispersed in a slow-release matrix comprising cross-linked high amylose starch; and a second release layer comprising an opioid in a second release matrix; and a third release layer, and b) a coat comprising the said core, wherein the said opioid is Tramadol

In at least one embodiment of the invention, includes a solid dosage formulation comprising a GABA analog and an opioid for release thereof over an extended period of time, the formulation comprises: a) a compressed core comprising at least two release layers wherein a first release layer comprising at least one GABA Analog dispersed in a slow-release matrix comprising cross-linked high amylose starch; and a second release layer comprising an opioid in a second release matrix; and a third release layer, and b) a coat comprising the said core, wherein the said opioid is morphine

In at least one embodiment of the invention, includes a solid dosage formulation comprising a GABA analog and an opioid for release thereof over an extended period of time, the formulation comprises: a) a compressed core comprising at least two release layers wherein a first release layer comprising at least one GABA Analog dispersed in a slow-release matrix comprising cross-linked high amylose starch; and a second release layer comprising an opioid in a second release matrix; and a third release layer, and b) a coat comprising the said core, wherein the said opioid is Faxeladol.

In at least one embodiment of the invention, includes a solid dosage formulation comprising a GABA analog and an opioid for release thereof over an extended period of time, the formulation comprises: a) a compressed core comprising at least two release layers wherein a first release layer comprising at least one GABA Analog dispersed in a slow-release matrix comprising cross-linked high amylose starch; and a second release layer comprising an opioid in a second release matrix; and a third release layer, and b) a coat comprising the said core, wherein the said opioid is Oxycodone.

In at least one embodiment of the invention, includes a solid dosage formulation comprising a GABA analog and an opioid for release thereof over an extended period of time, the formulation comprises: a) a compressed core comprising at least two release layers wherein a first release layer comprising at least one GABA Analog dispersed in a slow-release matrix comprising cross-linked high amylose starch; and a second release layer comprising an opioid in a second release matrix; and a third release layer, and b) a coat comprising the said core, The exemplary combinations include; Pregabalin as the GABA Analog and tapentadol as an opioid, Gabapentin as the GABA Analog and tapentadol as an opioid, Pregabalin as the GABA Analog and tramadol as an opioid, Gabapentin as the GABA Analog and tramadol as an opioid, Pregabalin as the GABA Analog and axomadol as an opioid, Gabapentin as the GABA Analog and axomadol as an opioid, Pregabalin as the GABA Analog and faxeladol as an opioid, Gabapentin as the GABA Analog and faxeladol as an opioid, Pregabalin as the GABA Analog and morphine as an opioid, Pregabalin as the GABA Analog and oxycodone as an opioid.

In at least one embodiment of the invention, includes a solid dosage formulation comprising a GABA analog and an opioid for release thereof over an extended period of time, the formulation comprises: a) a compressed core comprising at least two release layers wherein a first release layer comprising at least one GABA Analog dispersed in a slow-release matrix; and a second release layer comprising an opioid in a second release matrix; b) at least one permeable membrane pouch or a sachet comprising the said core; c) an encapsulating coat and exemplary GABA Analog is pregabalin, gabapentin, tiagabine and exemplary opioid is Axomadol, tapentadol, morphine, oxycodone, Tramadol and faxeladol.

In one embodiment, the dosage form uses an expansible and permeable membrane is used to house the core. The membrane can absorb body fluid, such as gastric juice, and can affect a slow and continuous release of controlled amounts of the GABA analog or opioid by means of diffusion or optionally by the use of osmosis. Suitable plastic or wax-like polymeric materials are especially hydrophilic materials such as methyl- or ethyl-cellulose, hydroxypropylcellulose, methyl- or ethyl-hydroxyethylcellulose, methyl- or ethyl-hydroxypropylcellulose, carboxymethylcellulose, polyvinyl acetate, polyvinylpyrrolidone, polyacrylonitrile, mixtures of polyvinylpyrrolidone with polyvinyl alcohol, resins based on phthalic acid anhydride/polyhydroxy alcohol, urethanes, polyamides, shellac, etc. The preferred are fully hydrolysed polyvinyl alcohol (more than 97%) is preferred. The membranes can be pre-formed pouch or a sachet.

Such membrane coated cores are provided with a disintegrating coat, upon contact with body fluids, is provided using suitable film coating materials. The covering materials include hydrophilic cellulose derivatives, such as cellulose ethers-methylcellulose, hydroxypropylcellulose or especially hydroxypropylmethylcellulose, mixtures of polyvinylpyrrolidone or of a copolymer of polyvinylpyrrolidone and polyvinyl acetate with hydroxypropylmethylcellulose, mixtures of shellac with hydroxypropylmethylcellulose, polyvinyl acetate or copolymers thereof with polyvinylpyrrolidone, or mixtures of water-soluble cellulose derivatives, such as hydroxypropylmethylcellulose, and water-insoluble ethyl cellulose can be used. It is also possible to substitute the covering with a hard gelatin capsules.

A further embodiment of this invention is to provide an oral dosage form that can be administered orally or rectal or sublingual or buccal.

Granules, spheroids, pellets, multiparticulates, capsules, patches tablets, sachets, controlled release suspensions, or in any other suitable dosage form incorporating such granules, spheroids, pellets or multiparticulates are also a part of the present invention.

### EXAMPLES

The following examples are shown for illustrating the invention related to a pharmaceutical composition comprising a GABA Analog, at least one opioid and at least one pharmaceutically acceptable excipient and the said dosage form is retained in the stomach for at least four hours and is suitable for once daily or twice daily administration. These examples in no way limit the scope of the invention. The person skilled in the art will know how the combination may be modified using other formulations and excipients.

### Manufacturing Process: Cross-Linked Amylose

The cross-linking of amylose is well known in the literature and the desired cross-linking of amylase can be carried out using the methods described in BIOCHIMIE 1978, 60, 535-537. The cross-linking of amylose is well-known in the literature. For example, the desired cross-linking can be controlled in the manner described by Mateescu et al. in Analytical Letters, 1985, 18, 79-91, by reacting amylose with epichlorohydrin in an alkaline medium. For Example; Cross-linked amylose is produced by reaction of amylose with a cross-linking agent such as epichlorohydrin, in an alkaline medium. Similarly, In the same manner, amylose can also be cross-linked with 2,3-dibromopropanol.

U.S. Pat. No. 5,456,921 discloses cross-linked amylose having a cross-linking degree ranging from 1 to 10 and is known to be particularly useful as a controlled release excipient for the preparation of tablets by direct compression It is also known (WO94/02121) that α-amylase can be incorporated into tablets made of cross-linked amylose in order to increase the dissolution rate of low soluble drugs.

Cross-linked amylose having a cross-linking degree of 6 to 30 is further known (WO94/21236) to be useful as a binder and/or disintegrant excipient for the preparation of tablets by direct compression. The binding properties of this product are reported to be definitively superior to starch. The quality of the binding and the controlled release properties of cross-linked amylose are closely related to the cross-linking degree and to the relative amount of amylose present in the starch used for the manufacture.

In all these patent and laid-open applications described above, a laboratory scale process of manufacture of cross-linked amylose is disclosed, which consists of reacting in a planetary mixer a product distributed by Sigma Chemicals, which is called amylose and consists of a corn starch containing more than 70% of amylose w/w, with epichlorohydrin in an alkaline medium. The obtained product is washed on a Buchner funnel with a solution of acetone and dried with pure acetone. About 40 Kg of acetone are needed to manufacture 1 Kg of cross-linked high amylose starch. It is well known in the art that the use of alcohols and/or acetone for the treatment of starch is reported to complex the amylose fraction.

Different degrees of cross-linking can be obtained by varying the ratio of epichlorohydrin to amylose in the reaction vessel. Tablets prepared by direct compression of a dry mixture of cross-linked amylose and a drug swell in solution and show a sustained release of the drug. Depending on the degree of cross-linking of the matrix, different degrees of swelling are obtained. Increasing the degree of cross-linking of amylose first generates an increase of drug-release time, followed by a decrease of drug-release time. The peak drug-release time is observed at a cross-linking degree value of 7.5. A further increase in the degree of cross-linking leads to an accelerated drug release from the cross-linked amylose tablets as a consequence of the erosion process. For cross-linking degree equal or greater than 7.5, increasing the degree of cross-linking of amylose generates a decrease of drug-release time. With degrees of cross-linking above 11, the swollen polymeric matrix presents in vitro disintegration over a period of approximately 90 minutes. The present inventors surprisingly found that a polymer of amylose cross-linked with a cross-linking agent selected from 2,3-dibromopropanol and epichlorohydrin, wherein from about 0.1 to about 10 g of cross-linking agent was used to cross-link 100 g of amylose, is suitable for preparing slow release formulations comprising a GABA Analog, at least one opioid, and at least one pharmaceutically acceptable excipient.

Illustrative Manufacturing Process: The process includes gelatinization, cross linking the gelatinized high amylase starch, removal of by-products and thermal treatment to obtain cross-linked amylase desired properties.

A slurry containing 1.2 KG of high amylase starch was prepared by mixing 2.65 KG of water and slurry was thoroughly mixed. To the slurry, 1.97 KG of sodium hydroxide solution at 11.9% w/w was introduced under. The gelatinization was carried out 50° C. for 20 minutes in a 200 L GOAVEC® crystallization tank. Under intensive stirring, 50 G of epichlorohydrin was introduced into the 1.2 KG of the gelatinized high amylose starch recovered in the previous step. The reaction was carried out at 50° C. for an hour. After reaction, the reaction medium was diluted with 5 KG of water at 60° C. and the mixture was neutralized with an acetic acid solution (37.5% w/w) to obtain a pH below 8. The neutralized product was diluted with 5 KG of water at 50° C. And cooled down and retained at 4° C. The product recovered from the previous step was diluted under agitation with 10 KG of water at 50° C. A diafiltration was realized with an ALFA-LAVAL© apparatus model UFS-6 equipped with 6 hollow fiber polysulfone membrane of 60 mils opening and surface of 25 square feet with pore sizes of 50000 Da. An average of 50 KG Kg of water at 50° C. was used to remove all the by-products such as sodium acetate. Then, the resulting product was concentrated up to 3.8% w/w by ultra filtration and the p. The recovered product was cooled down to 4° C. and was maintained at that temperature until the next step. As briefly discussed hereinabove, the properties of the prepared cross-linked high amylose starch that are required to make it useful as an excipient for drug controlled release are surprisingly dependent to the thermal treatment applied to the slurry just before spray drying. In order to demonstrate this dependency, cross-linked high amylose starch prepared as disclosed hereinabove was treated at different temperatures (100° C. to 50° C.) and the preferred temperature is 90° C. The cross linked amylase slurry prepared as above was heated to 90° C. at constant stirring for about 5 minutes. Then, the reactant was cooled down to 50° C. under stirring and spray dried at 3.8% of solids in a Niro spray dryer model P6.3 of water evaporating capacity of 50 KG/HOUR, equipped with a atomizer disc and having an inlet temperature of 300° C. and an outlet temperature of 120° C. The dry cross-linked amylose powder is a controlled release excipient suitable for preparing pharmaceutical dosage form comprising a GABA Analogue such pregabalin or gabapentin, an opioid such as morphine or tapentadol or oxycodone and at least one pharmaceutical excipient, wherein the dosage form is suitable for once daily or twice daily administration.

### Manufacturing Process: Core

### First Release Layer

The a pharmaceutical dosage form comprising: a) a core comprising at least two release layers wherein a first release layer comprising at least one GABA Analog dispersed in a slow-release matrix comprising cross-linked high amylose starch; and a second release layer comprising at least one opioid dispersed in a slow-release matrix; and b) a coat comprising the said core. In a specific embodiment, the matrix of the core is a cross-linked high amylose starch prepared according to the process described above.

The first-release matrix is formed by mixing the ingredients and then compressing the mixture to form the first-release matrix layer. The weight of the first-release matrix can be from about 10% to about 80%. In a particular embodiment described in this invention contains 150 mg pregabalin wherein the first-release matrix is about 26% of the total weight of the tablet. In yet another embodiment, a tablet contains 300 mg pregabalin wherein the first-release matrix makes up about 33% of the total weight of the tablet. In still yet another embodiment, a tablet contains 600 mg pregabalin wherein the first-release matrix contributes 33% to the total weight of the tablet. Particular embodiments of this invention include a first-release matrix containing pregabalin in which the first-release matrix contains between about 10% and 90% of the total pregabalin present in the tablet, e.g. the pregabalin is about 45% of the tablet total weight, or about 50% of the tablet total weight.

In specific embodiments, the matrix makes up between about 10% and about 90% by weight of the first-release matrix layer i.e., the ratio of the matrix of the first layer to the active ingredient of the first-release matrix layer (w/w) is between about 0.1 and about 10, or between about 0.2 and about 9, or between about 0.2 and about 8, or between about 0.3 and about 7, or between about 0.4 and about 6, or between about 0.5 and about 5, or between about 0.6 and about 4, or between about 0.7 and about 4 or between about 1 and about 4, or between about 1 and about 3 and about 1.5 and about 2.5.

Optionally the carriers or vehicles are known to those skilled in the art and are found, for example, in Remington's Pharmaceutical Sciences, 14^{th} Ed. (1970) can be optionally included in the core. These include other suitable binders, glidants, lubricants, dyes, sweetening, microcrystalline cellulose, starch, cross-linked starch, cross-linked poly(vinyl pyrrolidone), and sodium carboxymethyl cellulose; flavoring agents; coloring agents; binders; preservatives; surfactants or flavoring agents can also be included. Comprising a GABA Analog and at least one pharmaceutical excipient.

Example; Cross linked amylose prepared according to the process described above was mixed with colloidal silicon dioxide and passed through a #30 mesh screener. Similarly, cross linked amylose prepared according to the process described above was mixed with pregabalin, in a blender after passing through a #30 mesh screener. The Magnesium Stearate and Hydrogenated Vegetable Oil Type I are sieved through a #30 mesh screen separately and add to the blender. The cross linked amylose and colloidal silicon dioxide blend was blended with cross linked amylose-pregabalin blend and hydrogenated vegetable oil through a #30 mesh screen and add blend with other ingredients. This constitutes a first release layer.

### Second-Release Layer

This second-release matrix layer includes a physical mixture of polyvinyl acetate and polyvinylpyrrolidone and the active pharmaceutical ingredient(s) of the second-release matrix layer with tapentadol. The second-release matrix can also include a cross-linked high amylose starch prepared as described above and other optional components. The weight of the second-release matrix layer can be any percentage of the weight of the total composition between about 10% and about 90% such that it is between about 20% to about 90%, (w/w) of a tablet of the invention, or about 25% to about 90%, or about 30% to about 85%, or about 35% to about 85%, or about 40% to about 85%, or about 45% to about 85%, or about 45% to about 90%, or about 50% to about 90% or about 50% to about 85%, or about 55% to about 90%, or about 55% to about 85%, or about 55% to about 80%, or about 60% to about 90%, or about 60% to about 85%, or about 60% to about 80%, or about 60% to about 75%, or about 65% to about 90%, or about 65% to about 85%, or about 65% to about 80%, or about 65% to about 75%, or about 65% or about 70% or about 75%.

The weight percentage of the polyvinyl acetate/polyvinylpyrrolidone mixture in the second-release matrix layer can have a wide range of values. In particular the polyvinyl acetate/polyvinylpyrrolidone mixture can be from about 10 to about 90 wt. % of the second-release matrix layer, preferably from about 20 to about 80 wt. %, or about 30 to about 60 wt. %. In a particular embodiment of this invention, Kollidon© SR makes up from about 45% by weight of a second-release matrix that is about 31% by weight pregabalin and about 23% xanthan gum.

The weight ratio of polyvinyl acetate to polyvinylpyrrolidone in the polyvinyl acetate/polyvinylpyrrolidone mixture can be a wide range of values. Preferably, such ratio is between from about 6:4 and 9:1; more likely between from about 7:3 and 6:1, even more preferably about 8:2. The molecular weight of the polyvinyl acetate component in the polyvinyl acetate/polyvinylpyrrolidone mixture can have a wide range of values. For Example, the average molecular weight of the polyvinyl acetate is about 100 to about 10,000,000; or about 1,000 to about 1,000,000; or about 10,000 to about 1,000,000; or about 100,000 to about 1,000,000; or about 450,000. Similarly, the average molecular weight of the polyvinylpyrrolidone can be from about 100 to about 10,000,000; or from about 1,000 to about 1,000,000; or from about 5,000 to about 500,000; or from about 10,000 to about 100,000; or about 50,000.

The polyvinyl acetate and polyvinylpyrrolidone mixture can be prepared by a variety of processes as described the art well known to person skilled in pharmaceutical art. For example, it can be prepared by simply mixing powders of polyvinylpyrrolidone and polyvinyl acetate and other ingredients. In a preferred embodiment of this invention, such mixture is spray dried powder of a colloidal dispersion of polyvinyl acetate and polyvinylpyrrolidone solution. This admixture can also be added optionally stabilizers glidants etc. Optionally the carriers or vehicles are known to those skilled in the art and are found, for example binders, glidants, lubricants, dyes, sweetening, microcrystalline cellulose, starch, cross-linked starch, cross-linked poly(vinyl pyrrolidone), and sodium carboxymethyl cellulose; flavoring agents; coloring agents; binders; preservatives; surfactants or flavoring agents can also be included. Suitable binding agents for the present invention include, but are not limited to, plant extracts, gums, synthetic or natural polysaccharides, polypeptides, alginates, synthetic polymers, or a mixture thereof. There can be easily found in Remington's Pharmaceutical Sciences, 14.sup.th Ed. (1970). These include other suitable plant extracts to be used as gelling agents include, but are not limited to, agar, ispaghula, psyllium, cydonia, ceratonia or a mixture thereof. Suitable synthetic polymers to be used as gelling agents include, but are not limited to, carboxyvinyl polymer, polyvinyl alcohol, polyvinyl pyrrolidone, polyethelene oxide, polyethylene glycols, copolymers of ethylene oxide and propylene oxide and their copolymers or a mixture thereof. In a preferred embodiment of this invention, the gelling agent is a gum such as xanthan gum, guar gum, acacia gum, ghatti gum, karaya gum, tragacanth gum or a mixture thereof, PEO 7,000,000 and HPMC K100 M. In a most preferred embodiment of this invention, xanthan gum is use.

Place a portion of the KollidonR™ SR in a blender and mix with colloidal silicon dioxide, an opioid such as tapentadol, after passing through Kason Separator with a #30 mesh screener, Xanthan gum and hydrogenated vegetable oil Type 1 that is sieved through a #30 mesh screen were added to a blender and all ingredients were blended together. The magnesium stearate sieved through a #30 mesh screen and blended with other ingredients to prepare the second release layer.

This constitutes a second release layer.

### Third Release Layer (Optional);

The third release layer is optional and can be easily prepared according to procedures known in the art. The active agent, which is optional, swellable polymers, diluents and other additives may be mixed and further processed by either dry, wet granulation or direct compression. For Example, in one embodiment of this invention, Microcrystalline Cellulose, Crospovidone, Silicon Dioxide, Magnesium Stearate and Polyvinyl Pyyrolidone were mixed in a mixer and compressed into a third release layer. The third release layer can comprise an opioid such as tapentadol as an immediate release layer.

In another embodiment of this invention, a third layer is formed by mixing tapentadol, Microcrystalline Cellulose, Crospovidone, Silicon Dioxide, Magnesium Stearate and Polyvinyl Pyyrolidone in a mixer and compressed into a third release layer to form a an immediate release layer.

In another embodiment of this invention, oxycodone, Microcrystalline Cellulose, Crospovidone, Silicon Dioxide, Magnesium Stearate and Polyvinyl Pyyrolidone were mixed in a mixer compressed into a third release layer to form a an immediate release layer.

In another embodiment of this invention, axomadol, Microcrystalline Cellulose, Crospovidone, Silicon Dioxide, Magnesium Stearate and Polyvinyl Pyyrolidone were mixed in a mixer and compressed into a third release layer to form a an immediate release layer.

### Fourth Release Inert Layer (Optional);

The pharmaceutical dosage form of this invention may optionally comprise an inert layer. The inert layer constituents are easily discerned from the art. The inert layer devoid of any active agent, for example, may comprise of microcrystalline cellulose, magnesium stearate, Eudragit L100, and Poly Pyrrolidone. The inert layer is prepared by simple compression techniques known in the art and is used along with first, second and optionally third release layers to form a core as described below.

### Manufacturing Process: Core

The compositions for example, the first release layer, second release, a third release layer (which is optional) and an inert layer compositions are granulated, the granules of the individual layers are compressed to form a tablet using a rotary compression. When the compositions are processed by direct compression, the blends of the compositions respectively, may be compressed using a rotary press. The order of release layers is immaterial. For Example; in one embodiment of the invention, the first release layer comprising at least one GABA Analog dispersed in a slow-release matrix comprising cross-linked high amylose starch is compressed over a second release layer comprising an opioid dispersed in a slow-release matrix which in turn compressed over the inert layer. In another embodiment of this invention, the first release layer comprising at least one GABA Analog dispersed in a slow-release matrix comprising cross-linked high amylose starch, is compressed below a second release layer comprising an opioid dispersed in a slow-release matrix but above an inert layer. In another embodiment of this invention, the first release layer comprising at least one GABA Analog dispersed in a slow-release matrix comprising cross-linked high amylose starch, is compressed over a second release layer comprising an opioid dispersed in a slow-release matrix which in turn compressed over a third release layer, that optionally comprises either a GABA analog or an opioid or both and all three layers are compressed over an inert layer.

### Manufacturing Process: Coat

The coating of the core is carried out by techniques known in the art. The coating solution, according to respective formulation tables, was prepared using known emulsion polymerization techniques.

For Example, in one embodiment of the present invention, the coating solution is prepared by dissolving the pore forming agent in water and adding the dispersion of the water insoluble polymer to it, and then mixing the two together until the water soluble compound is dissolved in the aqueous dispersion. The coating composes solid content ranging from about 5% to about 25% w/w, preferably from about 10% to about 20%, more preferably from about 10% to about 15% w/w. The coating may be a film that may include water insoluble polymers such as ethyl cellulose, cellulose acetate, polyvinyl acetate, nitrocellulose, butadiene styrene copolymers, and water insoluble methacrylate copolymers. In some embodiments, Eudragit RS100, Eudragit RS PO, Eudragit RS 30D and Eudragit RS 12.5 may be used. The polymers that are insoluble below a pH of about 4.0 but soluble at pH above 7.0 are also used in another embodiment of invention. Such polymers include Eudragit L 100, Eudragit L 12.5, Eudragit 12.5 P, Eudragit L 30 D-55, Eudragit L 100-55, Eastacryl 30 D, Kollicoat MAE 30 D and Kollicoat MAE 30 D, cellulose acetate phthalate, hydroxypropyl methyl cellulose acetate succinate and the like and mixture thereof. Aqueous Ethyl cellulose in a dispersion form is used in a preferred embodiment

### Manufacturing Process: Membrane Pouch

Poly Vinyl Alcohol (EMD EMPROVE© Ph. Eur. USP) 10 g, water 90 mg and Glycerol 3.1 g were mixed and heated to 95′ c. The hot polyvinyl solution was cooled to room temperature and spread on a glass plate to form a thin soft flexible film of about 100 μm thickness was formed. The film was cut suitably to prepare pouch or a sachet of appropriate size depending on the size of the solid dosage form. The pouch was used encapsulate the coated compressed core.

The dosage form according to this invention comprises a coating surrounding the core. This can optionally comprise a passageway that can allow for controlled release of the drug from the core in a preferred embodiment and the exemplary passageways are well known and described, e.g., in U.S. Pat. Nos. 3,845,770; 3,916,899; 4,034,758; 4,077,407; 4,783,337 and 5,071,607. A passageway may be drilled so that upon contact with aqueous environment, water enters through the drilled passageways, swells the internal constituents and exerts pressure on that surface and eventually ruptures the coating from the drilled surface.

The diameter of the pore is selected such that no substantial delay occurs in rupturing of the coat. Preferably, the pore diameter is from about 500 m to about 1000 m. A pore forming agent may be defined herein as a solid or a liquid agent that forms micro porous coatings formed in situ by dissolution upon exposure to an aqueous environment of use. Pore forming-agents that may be used include, water-soluble compounds that have molecular weight of less than about 2000 daltons and hydrophilic polymers.

The pore forming agents that may be used in the present invention may be selected from the group consisting of alkali metal salts, alkaline earth metals, transition metal salts, organic compounds and the like. Exemplary pore forming material include alkali metal salts include, but not limited to, sodium chloride, sodium bromide, sodium carbonate, potassium chloride, potassium sulfate, potassium phosphate, sodium acetate, sodium citrate, potassium nitrate, and the like. Examples of alkaline earth metal salts include, but not limited to, calcium phosphate, calcium nitrate, calcium chloride, and the like. Examples of transition metal salts include, but not limited to, ferric chloride, ferrous sulfate, zinc sulfate, cupric chloride, manganese fluoride, manganese fluorosilicate, and the like. Examples of organic aliphatic oils include, but not limited to, diols and polyols, aromatic oils including diols and polyols, and other polyols such as polyhydric alcohol, polyalkylene glycol, polyglycol and the like. Even organic compounds that may be used as pore forming agent. The most preferred pore forming agent used in the coating composition of the present invention is selected from sugar alcohols, most preferably mannitol. Hydrophilic polymers may also be used as pore forming agents and they are selected from the group comprising of vinyl polymers, cellulose derivatives, polyethylene glycols and the like and mixtures thereof.

### Example 1

The pharmaceutical dosage form comprising 150 mg pregabalin and 50 mg Tapentadol at least one pharmaceutically acceptable excipient was prepared in accordance with the formula of Table 1 below;

TABLE 1

| | |
|---|---|
| mg | percent |

CORE

| | | |
|---|---|---|
| First Release Layer | | |
| Pregabalin | 75 | 62.46 |
| Hydrogenated Vegetable Oil | 0.7 | 0.58 |
| Silica | 0.18 | 0.15 |
| Cross Linked Amylose | 43.5 | 36.23 |
| Magnesium Stearate | 0.7 | 0.58 |
| Total First Release Layer | 120.08 | 100.00 |
| Second Release Layer | | |
| Pregabalin | 75 | 26.79 |
| Hydrogenated Vegetable Oil | 2.6 | 0.93 |
| Silica | 0.5 | 0.18 |
| Magnesium Stearate | 1.3 | 0.46 |
| Kollidon SR ® | 133.7 | 47.75 |
| Xanthan Gum | 66.9 | 23.89 |
| Total Second Release Layer | 280 | 100.00 |
| Third Release Layer | | |
| Tapentadol | 50 | 31.50 |
| Microcrystalline Cellulose | 50 | |
| Crospovidone | 40 | 25.20 |
| Colloidal Silicon Dioxide | 5.5 | 3.46 |
| Magnesium Stearate | 2.5 | 1.57 |
| Polyvinyl Pyrrolidone | 10 | 6.30 |
| Talc | 0.75 | 0.47 |
| Total Third Layer | 158.75 | 68.50 |
| Inert Layer | | |
| Microcrystalline Cellulose | 75 | 40.98 |
| Eudragit L100 | 50 | 27.32 |
| Polyvinyl Pyrrolidone | 53 | 28.96 |
| Magnesium Stearate | 2.5 | 1.37 |
| Talc | 2.5 | 1.37 |
| Total Inert Layer | 183 | 100.00 |
| TOTAL CORE | 400.08 | |

COAT

| | | |
|---|---|---|
| Ethyl Cellulose | 20 | 25.56 |
| Sodium Lauryl Sulfate | 0.75 | 0.96 |
| Aqucoat ECD | 50 | 63.90 |
| Cetyl Alcohol | 1.5 | 1.92 |
| Dibutyl Sebacate | 5 | 6.39 |
| Triethyl Citrate | 1 | 1.28 |
| Water | | |
| TOTAL COAT | 78.25 | 100.00 |
| TOTAL TABLET | 1037.16 | |

### Example 2

The pharmaceutical dosage form comprising 300 mg pregabalin and 100 mg of Tapentadol at least one pharmaceutically acceptable excipient was prepared according to the formula of Table 2 below;

TABLE 2

| | mg | percent |
|---|---|---|
| CORE | | |
| First Release Layer | | |
| Pregabalin | 150 | 62.48 |
| Hydrogenated Vegetable Oil | 1.35 | 0.56 |
| Silica | 0.36 | 0.15 |
| Cross Linked Amylose | 87 | 36.24 |
| Magnesium Stearate | 1.35 | 0.56 |
| Total First Release Layer | 240.06 | 100.00 |
| Second Release Layer | | |
| Pregabalin | 150 | 37.50 |
| Hydrogenated Vegetable Oil | 3.6 | 0.90 |
| Silica | 0.7 | 0.18 |
| Magnesium Stearate | 1.8 | 0.45 |
| Kollidon SR ® | 162.5 | 40.63 |
| Xanthan Gum | 81.4 | 20.35 |
| Total Second Release Layer | 400 | 100.00 |
| Third Release Layer | | |
| Tapentadol | 100 | 47.90 |
| Microcrystalline Cellulose | 50 | |
| Crospovidone | 40 | 19.16 |
| Colloidal Silicon Dioxide | 5.5 | 2.63 |
| Magnesium Stearate | 2.5 | 1.20 |
| Polyvinyl Pyrrolidone | 10 | 4.79 |
| Talc | 0.75 | 0.36 |
| Total Third Layer | 208.75 | 76.05 |
| Inert Layer | | |
| Microcrystalline Cellulose | 150 | 58.14 |
| Eudragit L100 | 50 | 19.38 |
| Polyvinyl Pyrrolidone | 53 | 20.54 |
| Magnesium Stearate | 2.5 | 0.97 |
| Talc | 2.5 | 0.97 |
| Total Inert Layer | 258 | 100.00 |
| TOTAL CORE | 640.06 | |
| COAT | | |
| Ethyl Cellulose | 20 | 25.56 |
| Sodium Lauryl Sulfate | 0.75 | 0.96 |
| Aqucoat ECD | 50 | 63.90 |
| Cetyl Alcohol | 1.5 | 1.92 |
| Dibutyl Sebacate | 5 | 6.39 |
| Triethyl Citrate | 1 | 1.28 |
| Water | | |
| TOTAL COAT | 78.25 | 100.00 |
| TOTAL TABLET | 1567.12 | |

### Example 3

The pharmaceutical dosage form comprising 150 mg pregabalin and 50 mg of Tapentadol and at least one pharmaceutically acceptable excipient was prepared according to the formula of Table 3 below;

TABLE 3

| | mg | percent |
|---|---|---|
| CORE | | |
| First Release Layer | | |
| Pregabalin | 75 | 62.46 |
| Hydrogenated Vegetable Oil | 0.7 | 0.58 |
| Silica | 0.18 | 0.15 |
| Cross Linked Amylose | 43.5 | 36.23 |
| Magnesium Stearate | 0.7 | 0.58 |
| Total First Release Layer | 120.08 | 100.00 |
| Second Release Layer | | |
| Pregabalin | 75 | 32.00 |
| Hydrogenated Vegetable Oil | 2.6 | 1.11 |
| Silica | 0.5 | 0.21 |
| Magnesium Stearate | 1.3 | 0.55 |
| Kollidon SR ® | 100 | 42.66 |
| Xanthan Gum | 55 | 23.46 |
| Total Second Release Layer | 234.4 | 100.00 |
| Third Release Layer | | |
| Microcrystalline Cellulose | 50 | 47.08 |
| Crospovidone | 40 | 37.66 |
| Colloidal Silicon Dioxide | 3.2 | 3.01 |
| Magnesium Stearate | 2 | 1.88 |
| Polyvinyl Pyrrolidone | 10 | 9.42 |
| Talc | 1 | 0.94 |
| Total Third Layer | 106.2 | 100.00 |
| Immediate Release Layer | | |
| Tapentadol | 50 | 47.62 |
| Povidone K 30 USP | 12 | 11.43 |
| Microcrystalline cellulose | 25 | 23.81 |
| Croscarmellose sodium | 15 | 14.29 |
| Magnesium Stearate | 3 | 2.86 |
| Water* | | 0.00 |
| Total Immediate Release Layer | 105 | 100.00 |
| Inert Layer | | |
| Microcrystalline Cellulose | 75 | 40.98 |
| Eudragit L100 | 50 | 27.32 |
| Polyvinyl Pyrrolidone | 53 | 28.96 |
| Magnesium Stearate | 2.5 | 1.37 |
| Talc | 2.5 | 1.37 |
| Total Inert Layer | 183 | 100.00 |
| TOTAL CORE | 354.48 | |
| COAT | | |
| Ethyl Cellulose | 20 | 25.56 |
| Sodium Lauryl Sulfate | 0.75 | 0.96 |
| Aqucoat ECD | 50 | 63.90 |
| Cetyl Alcohol | 1.5 | 1.92 |
| Dibutyl Sebacate | 5 | 6.39 |
| Triethyl Citrate | 1 | 1.28 |
| Water | | |
| TOTAL COAT | 78.25 | 100.00 |
| TOTAL TABLET | 998.41 | |

### Example 4

The pharmaceutical dosage form comprising 150 mg gabapentin and 50 mg Tapentadol and at least one pharmaceutically acceptable excipient was prepared according to the formula of Table 4 below;

TABLE 4

| | mg | percent |
|---|---|---|
| CORE | | |
| First Release Layer | | |
| Pregabalin | 150 | 62.40 |
| Hydrogenated Vegetable Oil | 0.7 | 0.29 |
| Silica | 0.18 | 0.07 |
| Cross Linked Amylose | 88 | 36.61 |
| Magnesium Stearate | 1.5 | 0.62 |
| Total First Release Layer | 240.38 | 100.00 |
| Second Release Layer | | |
| Tapentadol | 50 | 25.81 |
| Microcrystalline Cellulose | 130 | 67.10 |
| Crospovidone | 40 | 20.65 |
| Colloidal Silicon Dioxide | 5.5 | 2.84 |
| Magnesium Stearate | 2.5 | 1.29 |
| Polyvinyl Pyrrolidone | 15 | 7.74 |
| Talc | 0.75 | 0.39 |
| Total Second Release Layer | 193.75 | 100.00 |
| Membrane Pouch | | |
| Polyvinyl Alcohol | 223 | 82.90 |
| Glycerol | 46 | 17.10 |
| Total Covering | 269 | 100.00 |
| TOTAL CORE | 703.13 | |
| COAT | | |
| Ethyl Cellulose | 20 | 19.37 |
| Sodium Lauryl Sulfate | 0.75 | 0.73 |
| Aquacoat ECD | 75 | 72.64 |
| Cetyl Alcohol | 1.5 | 1.45 |
| Dibutyl Sebacate | 5 | 4.84 |
| Triethyl Citrate | 1 | 0.97 |
| Water | | |
| TOTAL COAT | 103.25 | 100.00 |
| TOTAL TABLET | 806.38 | |

### Example 5

The pharmaceutical dosage form comprising 300 mg pregabalin, 100 mg Tapentadol and at least one pharmaceutically acceptable excipient was prepared according to the formula of Table 5 below;

TABLE 5

| | mg | percent |
|---|---|---|
| CORE | | |
| First Release Layer | | |
| Pregabalin | 300 | 65.41 |
| Hydrogenated Vegetable Oil | 1.5 | 0.33 |
| Silica | 0.18 | 0.04 |
| Cross Linked Amylose | 155 | 33.79 |
| Magnesium Stearate | 2 | 0.44 |
| Total First Release Layer | 458.68 | 100.00 |
| Second Release Layer | | |
| Tapentadol | 100 | 49.32 |
| Microcrystalline Cellulose | 130 | 64.12 |
| Crospovidone | 50 | 24.66 |
| Colloidal Silicon Dioxide | 6.8 | 3.35 |
| Magnesium Stearate | 3.2 | 1.58 |
| Polyvinyl Pyrrolidone | 12 | 5.92 |
| Talc | 0.75 | 0.37 |
| Total Second Release Layer | 202.75 | 100.00 |
| Membrane Pouch | | |
| Polyvinyl Alcohol | 360 | 82.90 |
| Glycerol | 66 | 17.10 |
| Total Covering | 426 | 100.00 |
| TOTAL CORE | 1087.43 | |
| COAT | | |
| Ethyl Cellulose | 20 | 19.37 |
| Sodium Lauryl Sulfate | 0.75 | 0.73 |
| Aquacoat ECD | 75 | 72.64 |
| Cetyl Alcohol | 1.5 | 1.45 |
| Dibutyl Sebacate | 5 | 4.84 |
| Triethyl Citrate | 1 | 0.97 |
| Water | | |
| TOTAL COAT | 103.25 | 100.00 |
| TOTAL TABLET | 1190.68 | |

### Example 6

The pharmaceutical dosage form comprising 150 mg gabapentin and 50 mg of morphine at least one pharmaceutically acceptable excipient was prepared according to the formula of Table 6 below;

TABLE 6

| | mg | percent |
|---|---|---|
| CORE | | |
| First Release Layer | | |
| Gabapentin | 150 | 76.89 |
| Hydrogenated Vegetable Oil | 0.7 | 0.36 |
| Silica | 0.18 | 0.09 |
| Cross Linked Amylose | 43.5 | 22.30 |
| Magnesium Stearate | 0.7 | 0.36 |
| Total First Release Layer | 195.08 | 100.00 |
| Second Release Layer | | |
| Morphine | 50 | 19.61 |
| Hydrogenated Vegetable Oil | 2.6 | 1.02 |
| Silica | 0.5 | 0.20 |
| Magnesium Stearate | 1.3 | 0.51 |
| Kollidon SR ® | 133.7 | 52.43 |
| Xanthan Gum | 66.9 | 26.24 |
| Total Second Release Layer | 255 | 100.00 |
| Third Release Layer | | |
| Microcrystalline Cellulose | 90 | 51.21 |
| Crospovidone | 60 | 34.14 |
| Colloidal Silicon Dioxide | 7.5 | 4.27 |
| Magnesium Stearate | 2.5 | 1.42 |
| Polyvinyl Pyrrolidone | 15 | 8.53 |
| Talc | 0.75 | 0.43 |
| Total Third Layer | 175.75 | 100.00 |
| Membrane Pouch | | |
| Polyvinyl Alcohol | 223 | 82.90 |
| Glycerol | 46 | 17.10 |
| Total Covering | 269 | 100.00 |
| TOTAL CORE | 894.83 | |
| COAT | | |
| Ethyl Cellulose | 20 | 19.37 |
| Sodium Lauryl Sulfate | 0.75 | 0.73 |
| Aquacoat ECD | 75 | 72.64 |
| Cetyl Alcohol | 1.5 | 1.45 |
| Dibutyl Sebacate | 5 | 4.84 |
| Triethyl Citrate | 1 | 0.97 |
| Water | | |
| TOTAL COAT | 103.25 | 100.00 |
| TOTAL TABLET | 998.08 | |

### Example 7

The pharmaceutical dosage form comprising 150 mg Gabapentin and 5 mg of Oxycodone at least one pharmaceutically acceptable excipient was prepared according to the formula of Table 7 below;

TABLE 7

| | mg | percent |
|---|---|---|
| CORE | | |
| First Release Layer | | |
| Gabapentin | 150 | 76.89 |
| Hydrogenated Vegetable Oil | 0.7 | 0.36 |
| Silica | 0.18 | 0.09 |
| Cross Linked Amylose | 43.5 | 22.30 |
| Magnesium Stearate | 0.7 | 0.36 |
| Total First Release Layer | 195.08 | 100.00 |
| Second Release Layer | | |
| Oxycodone | 5 | 3.56 |
| Hydrogenated Vegetable Oil | 2 | 1.43 |
| Silica | 0.5 | 0.36 |
| Magnesium Stearate | 0.8 | 0.57 |
| Kollidon SR ® | 88 | 62.72 |
| Xanthan Gum | 44 | 31.36 |
| Total Second Release Layer | 140.3 | 100.00 |
| Third Release Layer | | |
| Microcrystalline Cellulose | 90 | 51.21 |
| Crospovidone | 60 | 34.14 |
| Colloidal Silicon Dioxide | 7.5 | 4.27 |
| Magnesium Stearate | 2.5 | 1.42 |
| Polyvinyl Pyrrolidone | 15 | 8.53 |
| Talc | 0.75 | 0.43 |
| Total Third Layer | 175.75 | 100.00 |
| Membrane Pouch | | |
| Polyvinyl Alcohol | 223 | 82.90 |
| Glycerol | 46 | 17.10 |
| Total Covering | 269 | 100.00 |
| TOTAL CORE | 780.13 | |
| COAT | | |
| Ethyl Cellulose | 20 | 19.37 |
| Sodium Lauryl Sulfate | 0.75 | 0.73 |
| Aquacoat ECD | 75 | 72.64 |
| Cetyl Alcohol | 1.5 | 1.45 |
| Dibutyl Sebacate | 5 | 4.84 |
| Triethyl Citrate | 1 | 0.97 |
| Water | | |
| TOTAL COAT | 103.25 | 100.00 |
| TOTAL TABLET | 883.38 | |

### Example 8

The pharmaceutical dosage form comprising 75 mg pregabalin and 50 mg of tapentadol at least one pharmaceutically acceptable excipient was prepared according to the formula of Table 8 below;

TABLE 8

| | mg | percent |
|---|---|---|
| CORE | | |
| First Release Layer | | |
| Pregabalin | 75 | 62.46 |
| Hydrogenated Vegetable Oil | 0.7 | 0.58 |
| Silica | 0.18 | 0.15 |
| Cross Linked Amylose | 43.5 | 36.23 |
| Magnesium Stearate | 0.7 | 0.58 |
| Total First Release Layer | 120.08 | 100.00 |
| Second Release Layer | | |
| Tapentadol | 50 | 31.86 |
| Microcrystalline Cellulose | 100 | 63.71 |
| Crospovidone | 40 | 25.49 |
| Colloidal Silicon Dioxide | 4.2 | 2.68 |
| Magnesium Stearate | 2 | 1.27 |
| Polyvinyl Pyrrolidone | 10 | 6.37 |
| Talc | 0.75 | 0.48 |
| Total Second Release Layer | 156.95 | 100.00 |
| Third Release Layer | | |
| Microcrystalline Cellulose | 90 | 51.21 |
| Crospovidone | 60 | 34.14 |
| Colloidal Silicon Dioxide | 7.5 | 4.27 |
| Magnesium Stearate | 2.5 | 1.42 |
| Polyvinyl Pyrrolidone | 15 | 8.53 |
| Talc | 0.75 | 0.43 |
| Total Third Layer | 175.75 | 100.00 |
| Membrane Pouch | | |
| Polyvinyl Alcohol | 223 | 82.90 |
| Glycerol | 46 | 17.10 |
| Total Covering | 269 | 100.00 |
| TOTAL CORE | 721.78 | |
| COAT | | |
| Ethyl Cellulose | 20 | 19.37 |
| Sodium Lauryl Sulfate | 0.75 | 0.73 |
| Aquacoat ECD | 75 | 72.64 |
| Cetyl Alcohol | 1.5 | 1.45 |
| Dibutyl Sebacate | 5 | 4.84 |
| Triethyl Citrate | 1 | 0.97 |
| Water | | |
| TOTAL COAT | 103.25 | 100.00 |
| TOTAL TABLET | 825.03 | |

### Example 9

The pharmaceutical dosage form comprising 150 mg pregabalin and 75 mg of tapentadol at least one pharmaceutically acceptable excipient was prepared according to the formula of Table 9 below;

TABLE 9

| | mg | percent |
|---|---|---|
| CORE | | |
| First Release Layer | | |
| Pregabalin | 150 | 68.94 |
| Hydrogenated Vegetable Oil | 0.7 | 0.32 |
| Silica | 0.18 | 0.08 |
| Cross Linked Amylose | 66 | 30.33 |
| Magnesium Stearate | 0.7 | 0.32 |
| Total First Release Layer | 217.58 | 100.00 |
| Second Release Layer | | |
| Tapentadol | 75 | 35.20 |
| Microcrystalline Cellulose | 135 | 63.37 |
| Crospovidone | 55 | 25.82 |
| Colloidal Silicon Dioxide | 5.1 | 2.39 |
| Magnesium Stearate | 2.2 | 1.03 |
| Polyvinyl Pyrrolidone | 15 | 7.04 |
| Talc | 0.75 | 0.35 |
| Total Second Release Layer | 213.05 | 100.00 |
| Third Release Layer | | |
| Microcrystalline Cellulose | 90 | 51.21 |
| Crospovidone | 60 | 34.14 |
| Colloidal Silicon Dioxide | 7.5 | 4.27 |
| Magnesium Stearate | 2.5 | 1.42 |
| Polyvinyl Pyrrolidone | 15 | 8.53 |
| Talc | 0.75 | 0.43 |
| Total Third Layer | 175.75 | 100.00 |
| Membrane Pouch | | |
| Polyvinyl Alcohol | 223 | 82.90 |
| Glycerol | 46 | 17.10 |
| Total Covering | 269 | 100.00 |
| TOTAL CORE | 875.38 | |
| COAT | | |
| Ethyl Cellulose | 24 | 20.47 |
| Sodium Lauryl Sulfate | 0.75 | 0.64 |
| Aquacoat ECD | 85 | 72.49 |
| Cetyl Alcohol | 1.5 | 1.28 |
| Dibutyl Sebacate | 5 | 4.26 |
| Triethyl Citrate | 1 | 0.85 |
| Water | | |
| TOTAL COAT | 117.25 | 100.00 |
| TOTAL TABLET | 992.63 | |

### Example 10

The pharmaceutical dosage form comprising 300 mg pregabalin and 100 mg of tapentadol at least one pharmaceutically acceptable excipient was prepared according to the formula of Table 10 below;

TABLE 10

| | mg | percent |
|---|---|---|
| CORE | | |
| First Release Layer | | |
| Pregabalin | 300 | 78.43 |
| Hydrogenated Vegetable Oil | 1 | 0.26 |
| Silica | 0.5 | 0.13 |
| Cross Linked Amylose | 80 | 20.92 |
| Magnesium Stearate | 1 | 0.26 |
| Total First Release Layer | 382.5 | 100.00 |
| Second Release Layer | | |
| Tapentadol | 100 | 37.11 |
| Microcrystalline Cellulose | 175 | 64.95 |
| Crospovidone | 65 | 24.12 |
| Colloidal Silicon Dioxide | 6 | 2.23 |
| Magnesium Stearate | 2.7 | 1.00 |
| Polyvinyl Pyrrolidone | 20 | 7.42 |
| Talc | 0.75 | 0.28 |
| Total Second Release Layer | 269.45 | 100.00 |
| Third Release Layer | | |
| Microcrystalline Cellulose | 90 | 51.21 |
| Crospovidone | 60 | 34.14 |
| Colloidal Silicon Dioxide | 7.5 | 4.27 |
| Magnesium Stearate | 2.5 | 1.42 |
| Polyvinyl Pyrrolidone | 15 | 8.53 |
| Talc | 0.75 | 0.43 |
| Total Third Layer | 175.75 | 100.00 |
| Membrane Pouch | | |
| Polyvinyl Alcohol | 223 | 82.90 |
| Glycerol | 46 | 17.10 |
| Total Covering | 269 | 100.00 |
| TOTAL CORE | 1096.7 | |
| COAT | | |
| Ethyl Cellulose | 28 | 22.00 |
| Sodium Lauryl Sulfate | 0.75 | 0.59 |
| Aquacoat ECD | 90 | 70.73 |
| Cetyl Alcohol | 1.5 | 1.18 |
| Dibutyl Sebacate | 6 | 4.72 |
| Triethyl Citrate | 1 | 0.79 |
| Water | | |
| TOTAL COAT | 127.25 | 100.00 |
| TOTAL TABLET | 1223.95 | |

### Example 11

The pharmaceutical dosage form comprising 100 mg gabapentin and 100 mg tramadol at least one pharmaceutically acceptable excipient was prepared according to the formula of Table 11 below;

TABLE 11

| | mg | percent |
|---|---|---|
| CORE | | |
| First Release Layer | | |
| Gabapentin | 50 | 52.59 |
| Hydrogenated Vegetable Oil | 0.7 | 0.74 |
| Silica | 0.18 | 0.19 |
| Cross Linked Amylose | 43.5 | 45.75 |
| Magnesium Stearate | 0.7 | 0.74 |
| Total First Release Layer | 95.08 | 100.00 |
| Second Release Layer | | |
| Pregabalin | 50 | 19.61 |
| Hydrogenated Vegetable Oil | 2.6 | 1.02 |
| Silica | 0.5 | 0.20 |
| Magnesium Stearate | 1.3 | 0.51 |
| Kollidon SR ® | 133.7 | 52.43 |
| Xanthan Gum | 66.9 | 26.24 |
| Total Second Release Layer | 255 | 100.00 |
| Third Release Layer | | |
| Tramadol | 100 | 47.90 |
| Microcrystalline Cellulose | 50 | |
| Crospovidone | 40 | 19.16 |
| Colloidal Silicon Dioxide | 5.5 | 2.63 |
| Magnesium Stearate | 2.5 | 1.20 |
| Polyvinyl Pyrrolidone | 10 | 4.79 |
| Talc | 0.75 | 0.36 |
| Total Third Layer | 208.75 | 76.05 |
| Inert Layer | | |
| Microcrystalline Cellulose | 75 | 40.98 |
| Eudragit L100 | 50 | 27.32 |
| Polyvinyl Pyrrolidone | 53 | 28.96 |
| Magnesium Stearate | 2.5 | 1.37 |
| Talc | 2.5 | 1.37 |
| Total Inert Layer | 183 | 100.00 |
| TOTAL CORE | 350.08 | |
| COAT | | |
| Ethyl Cellulose | 20 | 25.56 |
| Sodium Lauryl Sulfate | 0.75 | 0.96 |
| Aqucoat ECD | 50 | 63.90 |
| Cetyl Alcohol | 1.5 | 1.92 |
| Dibutyl Sebacate | 5 | 6.39 |
| Triethyl Citrate | 1 | 1.28 |
| Water | | |
| TOTAL COAT | 78.25 | 100.00 |
| TOTAL TABLET | 987.16 | |

### Example 12

The pharmaceutical dosage form comprising 300 mg gabapentin and 100 mg Axomadol and at least one pharmaceutically acceptable excipient was prepared according to the formula of Table 12 below;

TABLE 12

| | mg | percent |
|---|---|---|
| CORE | | |
| First Release Layer | | |
| Pregabalin | 150 | 68.94 |
| Hydrogenated Vegetable Oil | 0.7 | 0.32 |
| Silica | 0.18 | 0.08 |
| Cross Linked Amylose | 66 | 30.33 |
| Magnesium Stearate | 0.7 | 0.32 |
| Total First Release Layer | 217.58 | 100.00 |
| Second Release Layer | | |
| Pregabalin | 150 | 42.25 |
| Hydrogenated Vegetable Oil | 2.6 | 0.73 |
| Silica | 0.5 | 0.14 |
| Magnesium Stearate | 1.3 | 0.37 |
| Kollidon SR ® | 133.7 | 37.66 |
| Xanthan Gum | 66.9 | 18.85 |
| Total Second Release Layer | 355 | 100.00 |
| Third Release Layer | | |
| Axomadol | 100 | 47.90 |
| Microcrystalline Cellulose | 50 | |
| Crospovidone | 40 | 19.16 |
| Colloidal Silicon Dioxide | 5.5 | 2.63 |
| Magnesium Stearate | 2.5 | 1.20 |
| Polyvinyl Pyrrolidone | 10 | 4.79 |
| Talc | 0.75 | 0.36 |
| Total Third Layer | 208.75 | 76.05 |
| Inert Layer | | |
| Microcrystalline Cellulose | 75 | 40.98 |
| Eudragit L100 | 50 | 27.32 |
| Polyvinyl Pyrrolidone | 53 | 28.96 |
| Magnesium Stearate | 2.5 | 1.37 |
| Talc | 2.5 | 1.37 |
| Total Inert Layer | 183 | 100.00 |
| TOTAL CORE | 572.58 | |
| COAT | | |
| Ethyl Cellulose | 20 | 25.56 |
| Sodium Lauryl Sulfate | 0.75 | 0.96 |
| Aqucoat ECD | 50 | 63.90 |
| Cetyl Alcohol | 1.5 | 1.92 |
| Dibutyl Sebacate | 5 | 6.39 |
| Triethyl Citrate | 1 | 1.28 |
| Water | | |
| TOTAL COAT | 78.25 | 100.00 |
| TOTAL TABLET | 1432.16 | |

### Example 13

The pharmaceutical dosage form comprising 150 mg pregabalin, 50 mg of tapentadol and 5 mg memantine at least one pharmaceutically acceptable excipient was prepared according to the formula of Table 13 below;

TABLE 13

| | mg | percent |
|---|---|---|
| CORE | | |
| First Release Layer | | |
| Pregabalin | 150 | 68.94 |
| Hydrogenated Vegetable Oil | 0.7 | 0.32 |
| Silica | 0.18 | 0.08 |
| Cross Linked Amylose | 66 | 30.33 |
| Magnesium Stearate | 0.7 | 0.32 |
| Total First Release Layer | 217.58 | 100.00 |
| Second Release Layer | | |
| Tapentadol | 75 | 26.79 |
| Hydrogenated Vegetable Oil | 2.6 | 0.93 |
| Silica | 0.5 | 0.18 |
| Magnesium Stearate | 1.3 | 0.46 |
| Kollidon SR ® | 133.7 | 47.75 |
| Xanthan Gum | 66.9 | 23.89 |
| Total Second Release Layer | 280 | 100.00 |
| Third Release Layer | | |
| Memantine | 5 | 4.40 |
| Microcrystalline Cellulose | 50 | 43.96 |
| Crospovidone | 40 | 35.16 |
| Colloidal Silicon Dioxide | 5.5 | 4.84 |
| Magnesium Stearate | 2.5 | 2.20 |
| Polyvinyl Pyrrolidone | 10 | 8.79 |
| Talc | 0.75 | 0.66 |
| Total Third Layer | 113.75 | 100.00 |
| Inert Layer | | |
| Microcrystalline Cellulose | 75 | 40.98 |
| Eudragit L100 | 50 | 27.32 |
| Polyvinyl Pyrrolidone | 53 | 28.96 |
| Magnesium Stearate | 2.5 | 1.37 |
| Talc | 2.5 | 1.37 |
| Total Inert Layer | 183 | 100.00 |
| TOTAL CORE | 497.58 | |
| COAT | | |
| Ethyl Cellulose | 20 | 25.56 |
| Sodium Lauryl Sulfate | 0.75 | 0.96 |
| Aqucoat ECD | 50 | 63.90 |
| Cetyl Alcohol | 1.5 | 1.92 |
| Dibutyl Sebacate | 5 | 6.39 |
| Triethyl Citrate | 1 | 1.28 |
| Water | | |
| TOTAL COAT | 78.25 | 100.00 |
| TOTAL TABLET | 1187.16 | |

### Dissolution Studies

The dissolution profile of the Examples 1 and 2 prepared according to the tables in 1 and 2 are in Table 14. The present inventors found that in order to achieve a slow release profile of pregabalin over at least a twelve hour period following the administration of drug, the in vitro release rate preferably corresponds to the following rate of pregabalin released when measured with a USP Type I apparatus in 50 mM phosphate, pH 6.8, and stirring between 50 and 150 rpm; The dissolution profile exhibited by the pharmaceutical composition of instant invention is as follows;

between 10% and 40% of the pregabalin is released from the formulation between 0 and about 2 hours of measurement, between about 30% and 60% of the pregabalin is released from the formulation between 2 and about 7 hours of the measurement, between about 50% and 80% of the agent is released from the formulation between 7 and about 12 hours of measurement, and between about 80% and 100% of the pregabalin is released from the formulation after about 20 hours of measurement; or preferably

between 15% and 35% of the pregabalin is released from the formulation between at 2 hours of measurement, between about 40% and 60% of the pregabalin is released from the formulation between at 7 hours of the measurement, between about 60% and 80% of the pregabalin is released from the formulation at 12 hours of measurement, and between about 85% and 100% of pregabalin is released from the formulation after about 20 hours of measurement, or

between 20% and 40% of pregabalin is released from the formulation between at 2 hours of measurement, between about 40% and 60% of pregabalin is released from the formulation between at 7 hours of the measurement, between about 60% and 80% of pregabalin is released from the formulation at 12 hours of measurement, and between about 85% and 100% of pregabalin is released from the formulation after about 20 hours of measurement.

TABLE 14
Dissolution Profile Example 1 and 2

| Hours | Example 1 | Example 2 |
|---|---|---|
| 0 | 0 | 0 |
| 2 | 18 | 19 |
| 4 | 31 | 33 |
| 6 | 44 | 45 |
| 8 | 54 | 53 |
| 10 | 61 | 62 |
| 12 | 70 | 71 |
| 14 | 78 | 79 |
| 16 | 81 | 82 |

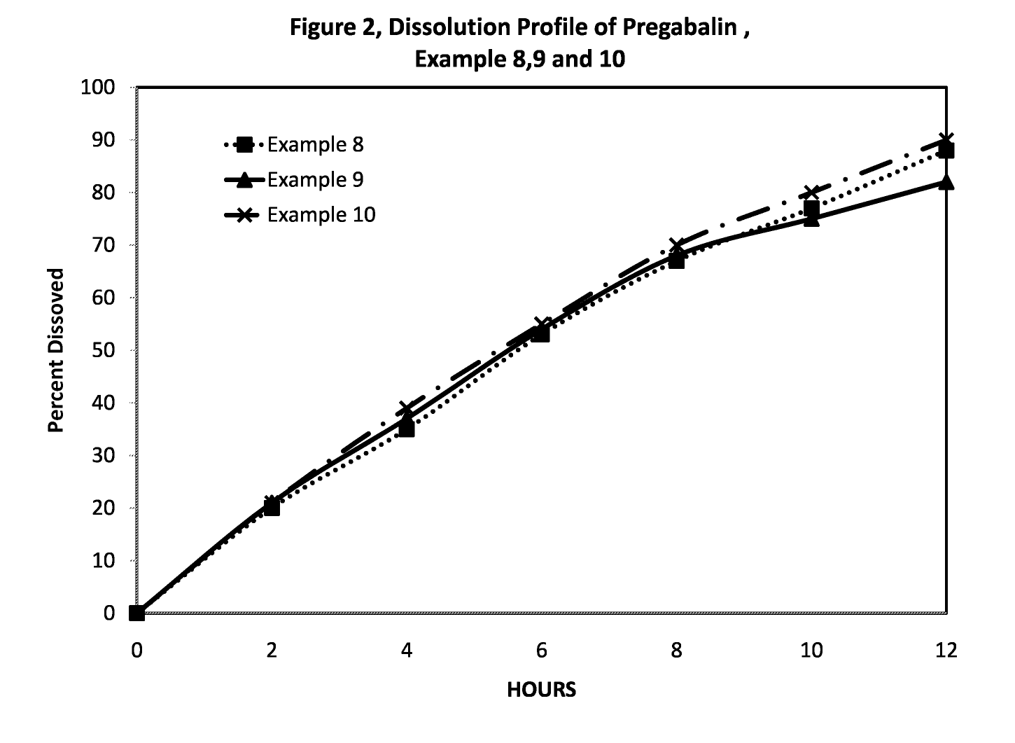

The Examples 8, 9 and 10 were subjected to the dissolution studies in vitro in simulated gastric fluids (0.1 N Hydrochloric acid at 100 rpm at 37° C. in a USP Type apparatus. The dissolution profile is below in FIG. 2;

### Bioavailability Studies;

Bioavailability studies were conducted to assess 1) Pharmacokinetic parameters from pregabalin, 2) Dose-proportionality between three dosage strengths (150 mg, 300 mg and 600 mg), 2) A comparative bioavailability study of 1×300 of Example 9 with 2×150 of Lyrica. A comparative bioavailability study of 200 mg dosage of the slow release composition of instant invention was compared to the commercial Lyrica.

A total of 20 subjects were enrolled in each study and all of them randomly received drugs as follows:

The studies were open, single dose or steady state, randomized, three-way cross-over design and each study included two treatment phases wherein each phase was separated by washout period of at least a 7 day wash-out period between each administration. Subjects were randomized to receive one of the above two regimens as randomly assigned by Latin Square and each subject crossed to each regimen according to the randomization sequence until all subjects have received all two regimens (with twenty one week separating each regimen). Blood samples were centrifuged within 2 hours of collection and the plasma were separated and frozen at −10° C. or lower until assayed. HPLC Analysis was carried out using stand techniques known to the person skilled in art.

### Dose Comparison

Table shows the GABA Analog (Pregabalin) plasma concentrations (ng/mL) of 75 mg (Example 8) dose 150 mg (Example 9) mg and dose 300 mg dose (Example 10) of a pharmaceutical dosage form comprising pregabalin and at least one pharmaceutically acceptable excipient of instant invention. The data establish that the dosages of Pregabalin in gastro-retentive dosage forms of examples 8, 9 and 10 are dose proportional with respect to the rate and extent of absorption of pregabalin.

### Clinical Study

Experimental data demonstrates a profound analgesic improvement between GABA Analog and opioid in treating pain. This may provide better pain relief with less opioid related side effects in clinical practice.

Purpose: This is a randomized, double blind, parallel arm study comparing a single dose of 1:1 fixed combination of pregabalin and tapentadol with pregabalin and tapentadol alone in patients with post-operative pain.

The primary objective of this randomized, double blind, parallel four arm study is to compare the analgesic effects of a fixed dose combination of pregabalin and tapentadol with pregabalin or tapentadol alone to determine additive activity of mu opioid analgesics in patients with post-operative pain.

A second goal is to further evaluate any side effects of the combination of pregabalin and tapentadol.

Study Type: Interventional

Study Design: Allocation: Randomized

Control: Active Control

Endpoint Classification: Efficacy Study

Intervention Model: Parallel Assignment

Masking: Double Blind (Subject, Investigator)

Primary Purpose: Treatment

Number of Patients: 40

Number of Arms: 3

Primary Outcome Measures: The primary goal of this study is to compare the analgesic effects of a fixed combination of pregabalin and tapentadol with pregabalin and tapentadol alone to determine additive or synergistic activity of mu opioid analgesics in patients with post-operative pain. [Time Frame: Time to the third request for the pain medication] [Designated as safety issue: No]

Secondary Outcome Measures: To determine if there are any side effects from the combination of morphine and methadone when given together. [Time Frame: assessed every 30 minutes] [Designated as safety issue: Yes]

| Arms | Assigned Interventions |
|---|---|
| 1: Active | Drug: Pregabalin |
| Comparator | Upon the 1st request for analgesic medication |
| Pregabalin | Pregabalin 150 mg, upon the 2nd request for analgesic |
| | medication Pregabalin 150 mg |
| 1: Active | Drug: Tapentadol HCl 50 mg |
| Comparator | Upon the 1st request for analgesic medication |
| Tapentadol | Tapentadol 75 mg, upon the 2nd request for analgesic |
| | medication Tapentadol 75 mg |
| 2: Experimental | Drug: Pregabalin (150 mg) + Tapentadol (75 mg) |
| Fixed dose | Upon the 1st request for analgesic medication |
| Pregabalin and | Pregabalin (150 mg) + Tapentadol (75 mg), upon |
| Tapentadol | the 2nd request for analgesic medication Pregabalin |
| | (150 mg) + Tapentadol (75 mg) |

### Inclusion and Exclusion Criteria:

Inclusion Criteria:

Retroperitoneal lymph node dissection

Planned post-operative analgesia with PCA at 1 mg continuous infusion and 1 mg every 10 minutes

18 years of age or older

English-speaking

Give informed consent to participate in this study

Exclusion Criteria:

Known hypersensitivity to Pregabalin or Tapentadol

Patients with past or present history of substance abuse

Patients with a long history of opioid treatment Patients with a history of chronic pain requiring daily analgesic use for more than 3 months

Patients treated with opioids within one month from the scheduled surgery

Creatinine clearance less than 50 mg/kg (using Cockcroft-Gault Equation)

Neurologic or psychiatric disease sufficient, in the doctor's opinion, to compromise data collection

MEASUREMENT METHOD: In the first 3 postoperative days, we evaluated the intensity of POP, anxiety, and pain relief with VASs. These scales consisted of horizontal lines graduated from 0 to 10, with 2 end points labeled on the front side: “no pain” to “worst pain,” “no anxiety” to “maximal anxiety,” and “no relief of pain” to “total relief of pain.” Patients were asked to quantify the level of pain and anxiety they were experiencing at the moment of the assessment, just before the analgesic administration. The pain relief was evaluated 45 minutes after the analgesic administration, with respect to the score of pain indicated just before this injection. However, the scores of POP and pain relief were so closely related that only POP is reported herein. The assessments of heart rate (radial pulse) and respiratory rate were performed by the nurses for 1 minute. Mean blood pressure (MBP) was measured with an automatics sphygmomanometer set around the arm, The first evaluation was performed when the patient had just recovered from anesthesia, immediately before he was taken from the recovery room, at the time defined as hour 0 (H₀). The assessments were then performed every 3 hours in the first 24 hours, and every 6 hours in the following 48 hours, that is, until the postoperative 72^{nd} hour (H₇₂). Overall, we collected 17 data points per patient for each postoperative criterion.

STATISTIC ALANALYSIS: The median rates of each parameter were determined at every hour of assessment. The hour corresponding to the highest median score could then be determined for all parameters. The earlier hour corresponding to a painless median level was also established. To prove that the differences between the scores indicated by patients were significant, depending on the post operative hour of assessment, then on parametric Friedman test was used. A value of P≦05 using the X² test was considered significant. The collected data were also plotted as a function of time for each patient; this allowed individual comparisons between rates of POP and other criteria in each patient. For these comparisons, non parametric Spearman correlation coefficients were used. 17 Correlation was considered significant when P less than 0.05.

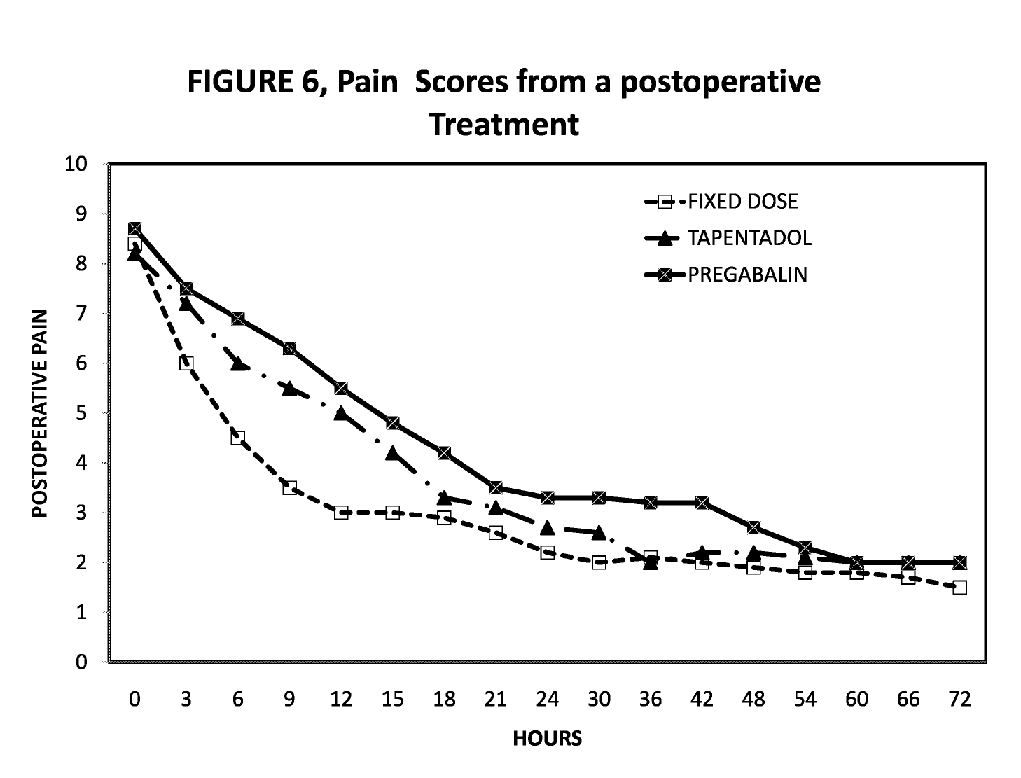

The FIG. 6 shows the post operative pain against period of measurement. The data clearly demonstrate that the dosage form of instant invention provides faster and better pain relief compared to the individual drugs.

What is claimed is:

1. A pharmaceutical dosage form comprising a therapeutically effective amount of at least one GABA Analog, at least one opioid, and at least one pharmaceutically acceptable excipient wherein the said dosage form is retained in the stomach for at least four hours.

2. A pharmaceutical dosage form according to claim 1, which is retained in the stomach for at least four hours, wherein between on an average 10% and 30% per hour of GABA Analog initially present at 0 hours, is released between 0 and 2 hours when tested in vitro using a USP Type I apparatus in 50 mM phosphate, pH 6.8, and stirring between 50 and 150 rpm.

3. A pharmaceutical dosage form according to claim 1, comprising: a) a core comprising at least two release layers wherein a first release layer comprising at least one GABA Analog, and a second release layer comprises at least one opioid and optionally at least one GABA Analog; and b) a coat comprising the said core.

4. A pharmaceutical a dosage form according to claim 1, comprising a) a compressed core comprising at least two release layers wherein a first release layer comprising at least one GABA Analog dispersed in a slow-release matrix; and a second release layer comprising an opioid in a second slow release matrix; b) at least one permeable membrane pouch comprising the said core; and c) an encapsulating coat.

5. A pharmaceutical dosage form according to claim 1, wherein the opioid is in immediate release form.

6. A pharmaceutical dosage form according to claim 1, wherein the opioid is in slow release form.

7. A pharmaceutical dosage form according to claim 1, comprising: a) a core comprising at least two layers, wherein a GABA Analog dispersed in at least one slow-release matrix; and a second release layer comprising at least one opioid dispersed in a second release matrix; and b) a coat comprising the said core, wherein between on an average 10% and 30% per hour of GABA Analog initially present at 0 hours, is released between 0 and 2 hours when tested in vitro using a USP Type I apparatus in 50 mM phosphate, pH 6.8, and stirring between 50 and 150 rpm.

8. A pharmaceutical dosage form according to claim 1, comprising: a) a core comprising at least two layers, wherein a GABA Analog dispersed in at least one slow-release matrix; and a second release layer comprising at least one opioid dispersed in a second release matrix; and b) a coat comprising the said core, wherein when tested in vitro using a USP Type I apparatus in 50 mM phosphate, pH 6.8, and stirring between 50 and 150 rpm, wherein
between 10% and 40% of GABA Analog released between 0 and about 2 hours of measurement,
between about 30% and 60% of GABA Analog released between 2 and about hours of the measurement,
between about 50% and 80% of GABA Analog released between 7 and about 12 hours of measurement, and
between about 80% and 100% of GABA Analog released after about 20 hours of measurement.

9. A pharmaceutical dosage form according to claim 1, wherein the release of GABA Analog is biphasic.

10. A pharmaceutical dosage form according to claim 3, wherein the second release layer comprises a physical mixture of polyvinyl acetate and polyvinylpyrrolidone.

11. A pharmaceutical dosage form according to claim 3, wherein the second release layer comprises a physical mixture of polyvinyl acetate, polyvinylpyrrolidone, a binder, GABA Analog; and wherein: the ratio of the first release layer/second release layer (w/w) is between from about 1.0 and to about 0.1.

12. A pharmaceutical dosage form according to claim 1, formulated for use for a period of every four hours, or every six hours, every eight hours, every twelve hours or every twenty-four hours.

13. A pharmaceutical dosage form according to claim 3, wherein either the first release layer or the second release layer or both prepared by compression.

14. A pharmaceutical dosage form according to claim 3, wherein the number of release layers is at least two and wherein the order of layers is immaterial.

15. A pharmaceutical dosage form according to claim 1, which comprises from about 5 to about 800 mg of GABA Analog, and from about 1 mg to about 500 mg of opioid.

16. A pharmaceutical dosage form according to claim 1, which, when administered to a patient in need thereof, provides a mean time to maximum plasma concentration (Tmax) of GABA Analog ranging from about four to about sixteen hours and the said dosage form is suitable once daily or twice daily administration.

17. A pharmaceutical dosage form according to claim 1, wherein said GABA analog is selected from the group consisting of pregabalin, gabapentin and tiagabine and said opioid is selected from the group consisting of axomadol, morphine, oxycodone, tapentadol, faxeladol and tramadol.

18. A pharmaceutical dosage form according to claim 1, wherein said GABA analog is selected from the group consisting of pregabalin, gabapentin and tiagabine and said opioid is selected from the group consisting of alfentanil, axomadol, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, faxeladol, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tapentadol and tramadol, and the dosage form is retained in the stomach for at least four hours and is suitable for once daily or twice daily administration.

19. A pharmaceutical dosage form according to claim 1, comprising a therapeutically effective amount of at least one GABA Analog, at least one opioid, and at least one pharmaceutically acceptable excipient wherein the said opioid is either in slow release form or in immediate release form and the dosage form is retained in the stomach for at least four hours.

20. A pharmaceutical dosage form according to claim 1, comprising a GABA analog, an Opioid and an NMDA receptor antagonist and at least one pharmaceutically acceptable excipient for treating pain and pain related disorders.

21. A process of preparing a pharmaceutical dosage form according to claim 1, said dosage from comprising: a) a core comprising at least two layers, wherein a GABA Analog dispersed in at least one slow-release matrix; and a second release layer comprising at least one opioid and optionally at least one GABA Analog dispersed in a second release matrix; and b) a coat comprising the said core, wherein said process comprises compressing the second release layer over a separately prepared said first release layer.

22. A method of treating a disorder comprising administering to a patient in need thereof an amount effective to treat said disorder of a pharmaceutical dosage form comprising a therapeutically effective amount of at least one GABA Analog, at least one opioid, and at least one pharmaceutically acceptable excipient wherein the said dosage form is retained in the stomach for at least four hours and is suitable for once daily or twice daily administration.

23. A method of treating a disorder according to claim 22, wherein the pharmaceutical a dosage form comprises: a) a core comprising at least two release layers wherein a first release layer comprising at least one GABA Analog, and a second release layer comprises at least one opioid and optionally at least one GABA Analog; and b) a coat comprising the said core, wherein the dosage form is retained in the stomach for at least four hours and is suitable for once daily or twice daily administration.

24. A method of treating a disorder according to claim 22, wherein the pharmaceutical a dosage form comprises a) a compressed core comprising at least two release layers wherein a first release layer comprising at least one GABA Analog dispersed in a slow-release matrix; and a second release layer comprising an opioid in a second slow release matrix; b) at least one permeable membrane pouch comprising the said core; c) an encapsulating coat and the said dosage form is suitable for once daily or twice daily administration.
